(19) United States
(12) Patent Application Publication
(10) Pub. No.: US 2009/0093501 A1
(43) Pub. Date: Apr. 9, 2009

(54) Heterocyclic antiviral compounds

(75) Inventors: Stephen Deems Gabriel, San Mateo, CA (US); Xiao-Fa Lin, Palo Alto, CA (US); Ferenc Makra, Palo Alto, CA (US); David Mark Rotstein, Sunnyvale, CA (US); Hanbiao Yang, Sunnyvale, CA (US)

Correspondence Address: Grant D. Green;Roche Palo Alto LLC, Patent Law Department, M/S A2-250, 3431 Hillview Avenue, Palo Alto, CA, 94304, US

(73) Assignee: Roche Palo Alto LLC

(21) Appl. No.: 12/284,039

(22) Filed: Sep. 18, 2008

Related U.S. Application Data
(60) Provisional application No. 60/994,426, filed on Sep. 19, 2007.
(60) Provisional application No. 61/084,724, filed on Jul. 30, 2008.

(51) Int. Cl. A61K 31/438; C07D 471/10; A61P 31/12; A61P 11/00; A61P 37/00; A61K 31/506; A61K 31/4545

(52) U.S. Cl. 514/256; 546/16; 544/230; 514/278

(57) ABSTRACT

This invention relates to piperidine derivatives of formula I wherein R¹, R², R³, R⁴ and Y are as defined herein useful in the treatment of a variety of disorders, including those in which the modulation of CCR5 receptors is implicated. Disorders that may be treated or prevented by the present derivatives include HIV and genetically related retroviral infections (and the resulting acquired immune deficiency syndrome, AIDS), rheumatoid arthritis, solid organ transplant reject (graft vs. host disease), asthma and COPR.

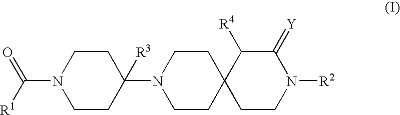

### CROSS REFERENCE TO PRIOR APPLICATIONS

This application claims the benefit of priority to U.S. Ser. No. 60/994,426 filed Sep. 19, 2007 and U.S. Ser. No. 61/084,724 filed Jul. 30, 2008 both of which are hereby incorporated in their entirety by reference.

### FIELD OF THE INVENTION

This invention relates to 3,9-diaza-spiro[5.5]undecane and 3,9-diaza-spiro[5.5]undecan-2-one compounds useful for the treatment of a variety of disorders in which modulation of the CCR5 receptor ligand binding is beneficial. More particularly, to new 9-(4-methyl-piperidin-4-yl)-3,9-diaza-spiro[5.5]undecane and 9-(4-methyl-piperidin-4-yl)-3,9-diaza-spiro[5.5]undecan-2-one compounds, to compositions containing said compounds and to uses of such derivatives. Disorders that may be treated or prevented by the present compounds include HIV-1 and genetically related retroviral infections (and the resulting acquired immune deficiency syndrome, AIDS), arthritis, asthma, chronic obstructive pulmonary disease (COPD) and rejection of transplanted organs.

### BACKGROUND OF THE INVENTION

Compounds of the present invention modulate the activity of the chemokine CCR5 receptors. The CCR5 receptor is a member of a subset of a large family chemokine receptors characterized structurally by two adjacent cysteine residues. Human chemokines include approximately 50 small proteins of 50-120 amino acids that are structurally homologous. (M. Baggiolini et al., Ann. Rev. Immunol. 1997 15:675-705) The chemokines are pro-inflammatory peptides that are released by a wide variety of cells such as macrophages, monocytes, eosinophils, neutrophiles, fibroblasts, vascular endothelial cells, smooth muscle cells, and mast cells, at inflammatory sites (reviewed in Luster, New Eng. J Med. 1998 338:436-445 and Rollins, Blood 1997 90:909-928). The name “chemokine”, is a contraction of “chemotactic cytokines”. The chemokines are a family of leukocyte chemotactic proteins capable of attracting leukocytes to various tissues, which is an essential response to inflammation and infection. Chemokines can be grouped into two subfamilies, based on whether the two amino terminal cysteine residues are immediately adjacent (CC family) or separated by one amino acid (CXC family). The CXC chemokines, such as interleukin-8 (IL-8), neutrophil-activating protein-2 (NAP-2) and melanoma growth stimulatory activity protein (MGSA) are chemotactic primarily for neutrophils and T lymphocytes, whereas the CC chemokines, such as RANTES (CCL5), MIP-1α (CCL3, macrophage inflammatory protein), MIP-1β (CCL4), the monocyte chemotactic proteins (MCP-1, MCP-2, MCP-3, MCP-4, and MCP-5) and the eotaxins (-1 and -2) are chemotactic for, among other cell types, macrophages, T lymphocytes, eosinophils, dendritic cells, and basophils. Naturally occurring chemokines that can stimulate the CCR5 receptor include MIP-1α, MIP-1β and RANTES.

Accordingly, drugs which inhibit the binding of chemokines such as MIP-1α, MIP-1β and RANTES to these receptors, e.g., chemokine receptor antagonists, may be useful as pharmaceutical agents which inhibit the action of chemokines such as MIP-1α, MIP-1β and RANTES on the target cells. The identification of compounds that modulate the function of CCR5 represents an excellent drug design approach to the development of pharmacological agents for the treatment of inflammatory conditions and diseases associated with CCR5 receptor.

The pharmacokinetic challenges associated with large molecules, proteins and peptides resulted in the establishment of programs to identify low molecular weight antagonists of CCR5. The efforts to identify chemokine modulators have been reviewed (W. Kazmierski et al. Biorg Med. Chem. 2003 11:2663-76; L. Agrawal and G. Alkhatib, Expert Opin. Ther. Targets 2001 5(3):303-326; Chemokine CCR5 antagonists incorporating 4-aminopiperidine scaffold, Expert Opin. Ther. Patents 2003 13(9):1469-1473; M. A. Cascieri and M. S. Springer, Curr. Opin. Chem. Biol. 2000 4:420-426, and references cited therein).

In U.S. Patent Publication 20050176703 published Aug. 11, 2005 S. D. Gabriel et al. disclosed 1-oxa-3,8-diaza-spiro[4.5]decan-2-one and 1-oxa-3,9-diaza-spiro[5.5]undecan-2-one derivatives which are CCR5 receptor antagonists.

### SUMMARY OF THE INVENTION

The present invention relates to a compound according to formula I and pharmaceutical compositions comprising a compound according to formula I admixed with at least one carrier, diluent or excipient wherein:

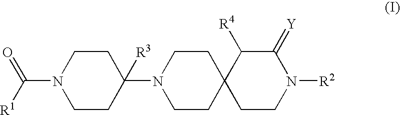

R¹ is selected from the group consisting of (i)-(v) and (vi):

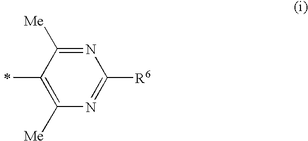

wherein:
   R⁶ is hydrogen, C₁₋₃ haloalkyl, —N(R^{a})-A¹-C(O)R⁷ or —NR^{b}R^{c};
   R^{a} is hydrogen or C₁₋₃ alkyl;
   A¹ is C₁₋₆ straight or branched alkylene;
   R^{b} and R^{c} are (A) together a group (CH₂)₂X¹(CH₂)₂, or, (B) independently R^{a} is hydrogen or C₁₋₃ alkyl and R^{b} is hydrogen, C₁₋₃ alkyl, C₁₋₃ acyl; —SO₂C₁₋₆ alkyl or C₁₋₆ hydroxyalkyl;

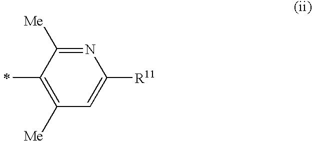

wherein:
   R¹¹ is hydrogen, C₃₋₆ cycloalkyl, cyano, OR^{6a} or —O-A¹-C(O)R⁷;
   A¹ is C₁₋₆ straight or branched alkylene;
   R^{6a} is hydrogen, C₁₋₃ alkyl or C₁₋₆ hydroxyalkyl;

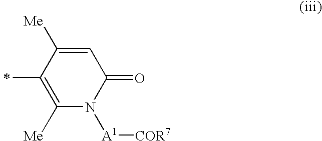

wherein:
   A¹ is C₁₋₆ straight or branched alkylene;

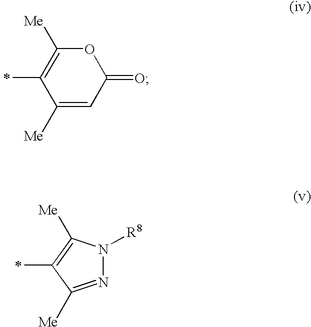

wherein:
   R⁸ is C₃₋₇ cycloalkyl, (CH₂)ₙCOR⁷, —OR¹⁵, heteroaryl selected from the group consisting of pyridine, pyrimidine, pyrazine and pyridazine said heteroaryl optionally substituted with C₁₋₃ alkyl or C₁₋₃ haloalkyl;
n is 1 to 3;
R¹⁵ is hydrogen or C₁₋₆ alkoxy;

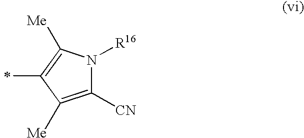

wherein:
R¹⁶ is hydrogen, hydroxyl or C₁₋₆ alkoxy;

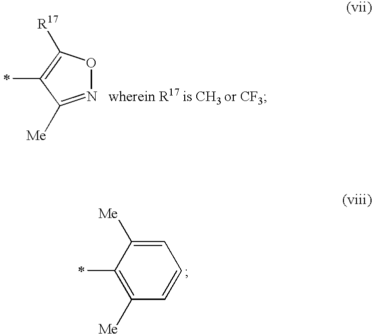

R² is A²-R⁹;

R³ is hydrogen or C₁₋₆ alkyl;

R⁴ is C₁₋₆ alkyl, C₁₋₆ alkoxy or phenyl;

R⁷ is hydroxy, C₁₋₆ alkoxy or NR^{e}R^{f};

A² is (CH₂)ₙ, C(O) or S(O)₂ wherein n is an integer from zero to three;

Y is O or H,H;

R⁷ is hydroxyl, NR^{e}R^{f}, or C₁₋₆ alkoxy;

R^{e} and R^{f} are (A) together a group (CH₂)₂X¹(CH₂)₂, or, (B) R^{e} and R^{f} are independently is hydrogen or C₁₋₃ alkyl;

R⁹ is: (a) C₃₋₆ cycloalkyl wherein said cycloalkyl is optionally substituted with one to three groups independently selected from the group consisting of OR¹⁴, C₁₋₃ alkyl, oxo, halogen and NR¹²R¹³ wherein R¹⁴ is hydrogen, C₁₋₆ alkyl, C₁₋₃ alkoxy-C₁₋₆ alkyl, carbamoyl, C1-3 alkylcarbamoyl or C₁₋₃ dialkylcarbamoyl, R¹² is C₁₋₆ alkylsulfonyl, C₁₋₆ alkoxycarbonyl or C₁₋₆ acyl and R¹³ is hydrogen or C₁₋₆ alkyl;

(b) heterocyclyl selected from the group consisting of tetrahydropyranyl, tetrahydrofuranyl, oxetanyl, [1,4]dioxanyl, 3-oxa-bicyclo[3.1.0]hex-6-yl or hexahydro-furo[2,3-b]furan-3-yl said heterocycle optionally substituted with one or two C₁₋₃ alkyl;

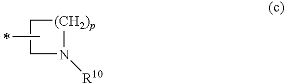

wherein;
p is an integer from one to three;
R¹⁰ is C₁₋₆ acyl, C₁₋₆ alkoxycarbonyl, C₁₋₆ alkyl-SO₂, C₁₋₆ haloalkyl, C₃₋₆ cycloalkyl;

(d) *—NR^{g}R^{h}
wherein:
   (A) R^{g} and R^{h} together are (CH₂)₂X¹(CH₂)₂, (B) together with the nitrogen to which they are attached are C₃₋₅ alkylene optionally substituted by C₁₋₃ alkyl, C₁₋₃ alkoxy, C₁₋₃ hydroxyalkyl or hydroxy, or, (C) taken independently R^{g} is hydrogen or C₁₋₃ alkyl and R^{h} is hydrogen, C₁₋₃ alkyl, C₁₋₃ acyl; —SO₂C₁₋₆alkyl or C₁₋₆ hydroxyalkyl;

(e) *—OR^{j} wherein R^{j} is C₁₋₆ alkyl or tetrahydropyran-4-yl;

(f) C₁₋₁₀ alkyl;

(g) C₁₋₁₀ heteroalkyl

(h) phenyl;

(i) pyridinyl;

(j) pyrazol-4-yl;

(k) imidazolyl;

wherein said phenyl, pyridinyl, pyrazol-4-yl or imidazolyl are optionally independently substituted with one to three groups independently selected from C₁₋₃ alkyl, C₁₋₃ alkoxy, C₃₋₆ cycloalkyl, halogen, C₁₋₆ alkoxycarbonyl, carbamoyl, C₁₋₆ alkyl carbamoyl, di-C₁₋₆ alkyl carbamoyl, C₁₋₆ alkylsulfanyl, C₁₋₆ alkylsulfinyl or C₁₋₆ alkylsulfonyl, amino, C₁₋₃ alkylamino or C₁₋₃ dialkylamino;

X¹ is O, S(O)ₘ, NR^{d};

R^{d} is hydrogen, C₁₋₃ alkyl, C₁₋₃ acyl or C₁₋₆ alkylsulfonyl;

m is zero to two; or,

pharmaceutically acceptable salts thereof.

The invention further provides a method for treating or preventing an human immunodeficiency virus (HIV-1) infection, or treating AIDS or ARC, in a patient in need thereof by administering a compound of formula I either alone, or in combination with other anti-HIV-1 drugs. The invention further provides a method for treating rheumatoid arthritis and inflammatory disorders, organ transplant rejection, COPD and asthma using a compound of formula I either alone or in combination with other drugs.

### DETAILED DESCRIPTION OF THE INVENTION

The phrase “a” or “an” entity as used herein refers to one or more of that entity, for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or [symbol] drawn through a bond each refer to the point of attachment of a functional group or other chemical moiety to the rest of the molecule of which it is a part. Thus, for example:

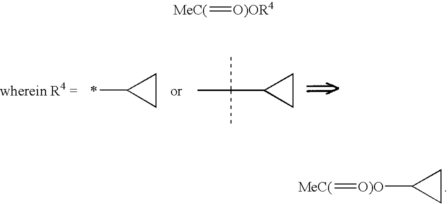

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

In one embodiment of the present invention there is provided a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In another embodiment of the present invention there is provided a compound according to formula I wherein R¹ is (i)-(v) or (vii) and R⁸ is C₃₋₇ cycloalkyl, (CH₂)ₙCOR⁷, heteroaryl selected from the group consisting of pyridine, pyrimidine, pyrazine and pyridazine said heteroaryl optionally substituted with C₁₋₃ alkyl or C₁₋₃ haloalkyl; and when R⁹ is (a) said cycloalkyl is optionally substituted with one to three groups independently selected from the group consisting of hydroxyl, C₁₋₆ alkoxy, C₁₋₃ alkyl, oxo and halogen.

In a second embodiment of the present invention there is provided a compound according to formula I R¹ is (i) and R⁶ is hydrogen or (ii) and R¹¹ is cyano; R³ is methyl; R⁴ is n-C₄H₉; A² is SO₂; and, R⁹ is C₁₋₆ alkyl, C₃₋₆ cycloalkyl, phenyl, pyridinyl, tetrahydropyranyl or NR^{g}R^{h}.

In another embodiment of the present invention there is provided a compound according to formula I R¹ is (i) and R⁶ is hydrogen or (ii) and R¹¹ is cyano; R³ is methyl; R⁴ is C₁₋₆ alkyl; A² is SO₂; and, R⁹ is C₁₋₆ alkyl, C₃₋₆ cycloalkyl, phenyl, pyridinyl, tetrahydropyranyl or NR^{g}R^{h}.

In a third embodiment of the present invention there is provided a compound according to formula I R¹ is (i) and R⁶ is hydrogen or (ii) and R¹¹ is cyano; R³ is methyl; R⁴ is n-C₄H₉; A² is SO₂; and, R⁹ is methyl, cyclopropyl, phenyl, 2-pyridinyl or NR^{g}R^{h}; and, R^{g} and R^{h} are independently are hydrogen or C₁₋₃ alkyl or together are (CH₂)₂X¹(CH₂)₂.

In a fourth embodiment of the present invention there is provided a compound according to formula I R¹ is (i) and R⁶ is hydrogen or (ii) and R¹¹ is cyano; R³ is methyl; R⁴ is n-C₄H₉; A² is SO₂; and, R⁹ is NR^{g}R^{h}; R^{g} and R^{h} are independently are hydrogen or C₁₋₃ alkyl or together are (CH₂)₂X¹(CH₂)_{2D}; and X¹ is O.

In a fifth embodiment of the present invention there is provided a compound according to formula I R¹ is (i) and R⁶ is hydrogen or (ii) and R¹¹ is cyano; R³ is methyl; R⁴ is n-C₄H₉; A² is SO₂; and, R⁹ is:

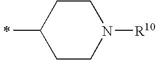

wherein R¹⁰ is C₁₋₆ acyl, C₁₋₆ alkoxycarbonyl, C₁₋₆ alkyl-SO₂, C₁₋₆ haloalkyl.

In a sixth embodiment of the present invention there is provided a compound according to formula I R¹ is (i) and R⁶ is hydrogen or (ii) and R¹¹ is cyano; R³ is methyl; R⁴ is n-C₄H₉; A² is CH₂; and, R⁹ is tetrahydropyran-4-yl, 4-C₁₋₆ alkoxy-cyclohexyl, 4-fluorophenyl or:

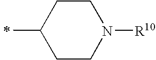

wherein R¹⁰ is C₁₋₆ acyl, C₁₋₆ alkoxycarbonyl, C₁₋₆ alkyl-SO₂, C₁₋₆ haloalkyl.

In a seventh embodiment of the present invention there is provided a compound according to formula I R¹ is (i) and R⁶ is hydrogen or (ii) and R¹¹ is cyano; R³ is methyl; R⁴ is n-C₄H₉; A² is C(O); and, R⁹ is tetrahydropyran-4-yl or 4-oxaq-tetrahydropyran.

In an eighth embodiment of the current invention there is provided a compound selected from TABLE 1.

In a ninth embodiment of the present invention there is provided a method for treating or preventing an human immunodeficiency virus (HIV-1) infection, or treating AIDS or ARC, in a patient in need thereof which comprises administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In a tenth embodiment of the present invention there is provided a method for treating or preventing an human immunodeficiency virus (HIV-1) infection, or treating AIDS or ARC, in a patient in need thereof which comprises co-administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above together with one or more compound(s) selected from the group consisting of HIV-1 nucleoside reverse transcriptase inhibitors, non-nucleoside reverse transcriptase inhibitors, HIV-1 protease inhibitors and HIV-1 viral fusion inhibitors.

In a eleventh embodiment of the present invention there is provided a method for treating an inflammatory disorder, in a patient in need thereof which comprises administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In a twelfth embodiment of the present invention there is provided a method for treating rheumatoid arthritis, in a patient in need thereof which comprises administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In a thirteenth embodiment of the present invention there is provided a method for treating rheumatoid arthritis, in a patient in need thereof which comprises co-administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above together with one or more anti-inflammatory or analgesic compounds.

In a fourteenth embodiment of the present invention there is provided a method for treating asthma or COPD, in a patient in need thereof which comprises administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In a fifteenth embodiment of the present invention there is provided a method for treating solid organ transplant rejection, in a patient in need thereof which comprises administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above.

In a sixteenth embodiment of the present invention there is provided a method for treating solid organ transplant rejection, in a patient in need thereof which comprises co-administering to the patient in need thereof a therapeutically effective amount of a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above together with one or more anti-rejection drugs or immunomodulators

In a seventeenth embodiment of the present invention there is provided a pharmaceutical composition comprising a compound according to formula I wherein R¹, R², R³, R⁴, R⁵, R⁶, R^{6a}, R⁷, R⁸, R⁹, R¹⁰, R¹¹, R¹², R¹³, R¹⁴, R¹⁵, R¹⁶, R¹⁷, R^{a}, R^{b}, R^{c}, R^{d}, R^{e}, R^{f}, R^{g}, R^{h}, R^{j}, A¹, A², X¹, Y, m, n and p are as defined herein above together with one or more carriers, excipients or diluents.

The definitions described herein may be appended to form chemically relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₆ alkyl” as used herein refers to an alkyl composed of 1 to 6 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 1 to 6 carbon. atoms. The C₁ acyl group is a formyl group wherein the one carbon atom is the carbonyl and R═H. The term “arylcarbonyl” as used herein means a group of formula C(═O)R wherein R is an aryl group; the term “benzoyl” as used herein an “arylcarbonyl” group wherein R is phenyl.

The terms “hydroxyalkyl” and “alkoxyalkyl” as used herein denotes alkyl radical as herein defined wherein one to three hydrogen atoms on different carbon atoms is/are replaced by hydroxyl or alkoxy groups respectively. A C₁₋₃ alkoxy-C₁₋₆ alkyl moiety refers to a C₁₋₆ alkyl substituent in which 1 to 3 hydrogen atoms are replaced by a C₁₋₃ alkoxy and the point of attachment of the alkoxy is the oxygen atom.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The terms “alkylsulfonyl” and “arylsulfonyl” as used herein denotes a group of formula —S(═O)₂R wherein R is alkyl or aryl respectively and alkyl and aryl are as defined herein.

The terms “alkylsulfinyl” and “arylsulfinyl” as used herein denotes a group of formula —S(═O)R wherein R is alkyl or aryl respectively and alkyl and aryl are as defined herein

The term “carbamoyl, C₁₋₆ alkyl carbamoyl and C₁₋₆ dialkyl carbamoyl refers to a moiety —C(O)NR′R″ where in R′=R″=H, R′=H/R″=C₁₋₆ alkyl and R′=R″=C₁₋₆ alkyl, respectively.

The terms “alkoxycarbonyl” and “aryloxycarbonyl” as used herein denotes a group of formula —C(═O)OR wherein R is alkyl or aryl respectively and alkyl and aryl are as defined herein.

The term “heteroalkyl” as used herein means an alkyl radical as defined herein wherein one, two or three hydrogen atoms have been replaced with a substituent independently selected from the group consisting of —OR^{a}, —NR^{b}R^{c}, and —S(O)ₙR^{d} (where n is an integer from 0 to 2), with the understanding that the point of attachment of the heteroalkyl radical is through a carbon atom, wherein R^{a} is hydrogen, acyl, alkyl, cycloalkyl, or cycloalkylalkyl; R^{b} and R^{c} are independently of each other hydrogen, acyl, alkyl, cycloalkyl, or cycloalkylalkyl; and when n is 0, R^{d} is hydrogen, alkyl, cycloalkyl, or cycloalkylalkyl, and when n is 1 or 2, R^{d} is alkyl, cycloalkyl, cycloalkylalkyl, amino, acylamino, or alkylamino. Representative examples include, but are not limited to, 2-hydroxyethyl, 3-hydroxypropyl, 2-hydroxy-1-hydroxymethylethyl, 2,3-dihydroxypropyl, 1-hydroxymethylethyl, 3-hydroxybutyl, 2,3-dihydroxybutyl, 2-hydroxy-1-methylpropyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like.

The terms “pyridazine”, “pyrimidine” and “pyrazine” refer to six-membered aromatic ring containing two nitrogen atoms which are ortho, meta and para, respectively.

The terms “pyrazol-4-yl”, “imidazolyl”, (iii) terahydropyranyl, (iv) tetrahydrofuranyl and (v) 4-oxa-tetrahydropyran refer to groups (i)-(iv) respectively in which any open valence can be substituted:

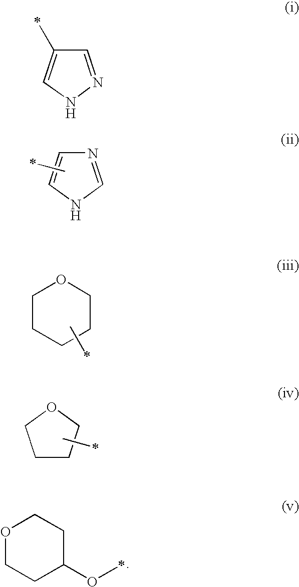

The terms, 3-oxa-bicyclo[3.1.0]hex-6-yl, hexahydro-furo[2,3-b]furan-3-yl and [1,4]dioxanyl refer to the moieties (vi). (vii) and (viii) respectively.

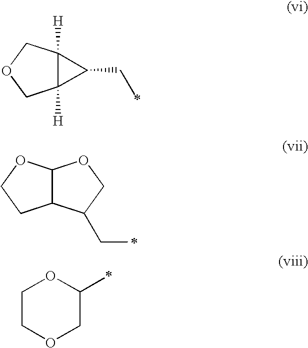

The term “alkylthio” or “alkylsulfanyl” means an —S-alkyl group, wherein alkyl is as defined above such as meththio, ethylthio, n-propylthio, i-propylthio, n-butylthio, hexylthio, including their isomers. “Lower alkylthio” or “lower thioalkyl” as used herein denotes an alkylthio group with a “lower alkyl” group as previously defined. “C₁₋₁₀ alkylthio” as used herein refers to an —S-alkyl wherein alkyl is C₁₋₁₀. “Arylthio” means an —S-aryl group, wherein aryl is as defined herein. “Phenylthio” is an “arylthio” moiety wherein aryl is phenyl. The terms “amino”, “alkylamino” and “dialkylamino” as used herein refer to —NH₂, —NHR and —NR₂ respectively and R is alkyl as defined above. The two alkyl groups attached to a nitrogen in a dialkyl moiety can be the same or different. The terms “aminoalkyl”, “alkylaminoalkyl” and “dialkylaminoalkyl” as used herein refer to NH₂(CH₂)ₙ—, RHN(CH₂)ₙ—, and R₂N(CH₂)ₙ— respectively wherein n is 1 to 6 and R is alkyl as defined above. “C₁₋₁₀ alkylamino” as used herein refers to an -aminoalkyl wherein alkyl is C₁₋₁₀. The term “phenylamino” as used herein refers to —NHPh wherein Ph represents an optionally substituted phenyl group.

The term “halogen” or “halo” as used herein means fluorine, chlorine, bromine, or iodine.

The term “alkylating agent” refers to a compound RZ¹ wherein Z¹ is a leaving group such as halo, C₁₋₄ alkanesulphonyloxy, benzenesulphonyloxy or p-toluenesulphonyloxy. Structural limitations on the R required for efficient alkylation are well known in the art.

### Methods for Treating HIV-1 Infections

HIV-1 infects cells of the monocyte-macrophage lineage and helper T-cell lymphocytes by exploiting a high affinity interaction of the viral enveloped glycoprotein (Env) with the CD-4 antigen. The CD-4 antigen was found to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor, were subsequently found to be co-receptors along with CD4 which are required for infection of cells by the human immunodeficiency virus (HIV). The central role of CCR5 in the pathogenesis of HIV was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly common in exposed/uninfected individuals suggesting the role of CCR5 in HIV cell entry (R. Liu et al., Cell 1996 86(3):367-377; M. Samson et al., Nature 1996 382(6593):722-725). The CD-4 binding site on the gp120 of HIV appears to interact with the CD4 molecule on the cell surface resulting in a conformational change that allows it to bind to either the CCR5 and/or CXCR-4 cell-surface receptor. This brings the viral envelope closer to the cell surface and allows interaction between gp41 on the viral envelope and a fusion domain on the cell surface, fusion with the cell membrane, and entry of the viral core into the cell. Accordingly, an agent which could block chemokine receptors in humans who possess normal chemokine receptors should prevent infection in healthy individuals and slow or halt viral progression in infected patients.

RANTES and an analog chemically modified on the N-terminus, aminooxypentane RANTES, were found to block HIV entry into the cells. (G. Simmons et al., Science 1997 276:276-279). Maraviroc (brand-named Selzentry, or Celsentri outside the U.S., CASRN 376348-65-1) is a CCCR5 chemokine receptor antagonist drug developed by the drug company Pfizer which has been approved for treatment of HIV-1 infections by the Y.S. Food and Drug Administration.

A-M. Vandamme et al. (Antiviral Chem. & Chemother., 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. While HAART has dramatically altered the prognosis for HIV infected persons, there remain many drawbacks to the current therapy including highly complex dosing regimes and side effects which can be very severe (A. Carr and D. A. Cooper, Lancet 2000 356(9239): 1423-1430). Moreover, these multidrug therapies do not eliminate HIV-1 and long-term treatment usually results in multidrug resistance, thus limiting their utility in long-term therapy. Development of new therapeutics which can be used in combination with NRTIs, NNRTIs, PIs and viral fusion inhibitors to provide better HIV-1 treatment remains a priority.

Typical suitable NRTIs for HAART therapy include zidovudine (AZT; RETROVIR®); didanosine (ddI; VIDEX®); zalcitabine (ddC; HIVID®); stavudine (d4T; ZERIT®); lamivudine (3TC; EPIVIR®); abacavir (ZIAGEN®); adefovir dipivoxil [bis-(POM)-PMEA; PREVON®]; lobucavir (BMS-180194), a nucleoside reverse transcriptase inhibitor disclosed in EP-0358154 and EP-0736533; BCH-10652, a reverse transcriptase inhibitor (in the form of a racemic mixture of BCH-10618 and BCH-10619) under development by Biochem Pharma; emitricitabine [(−)-FTC] in development by Triangle Pharmaceuticals; β-L-FD4 (also called β-L-D4C and named β-L-2′,3′-dicleoxy-5-fluoro-cytidene) licensed Vion Pharmaceuticals; DAPD, the purine nucleoside, (−)-β-D-2,6-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor under development by U.S. Bioscience Inc.

Typical suitable NNRTIs include nevirapine (BI-RG-587; VIRAMUNE®); delaviradine (BHAP, U-90152; RESCRIPTOR®); efavirenz (DMP-266; SUSTIVA®); PNU-142721, a furopyridine-thio-pyrimidine under development by Pfizer; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H, 3H)-pyrimidinedione); and (+)-calanolide A (NSC-675451) and B, coumarin derivatives disclosed in U.S. Pat. No. 5,489,697.

Typical suitable PIs include saquinavir (Ro 31-8959; INVIRASE®; FORTOVASE®); ritonavir (ABT-538; NORVIR®); indinavir (MK-639; CRIXIVAN®); nelfnavir (AG-1343; VIRACEPT®); amprenavir (141W94; AGENERASE®); lasinavir (BMS-234475); DMP-450, a cyclic urea under development by Triangle Pharmaceuticals; BMS-2322623, an azapeptide under development by Bristol-Myers Squibb as a 2nd-generation HIV-1 PI; ABT-378 under development by Abbott; and AG-1549 an imidazole carbamate under development by Agouron Pharmaceuticals, Inc.

Other antiviral agents include hydroxyurea, ribavirin (1-β-D-ribofuranosyl-1H-1,2,4-triazole-3-carboxamide), IL-2, IL-12, pentafuside. Hydroxyurea (Droxia), a ribonucleoside triphosphate reductase inhibitor shown to have a synergistic effect on the activity of didanosine and has been studied with stavudine. IL-2 (aldesleukin; PROLEUKIN®) is disclosed in Ajinomoto EP-0142268, Takeda EP-0176299, and Chiron U.S. Pat. No. RE 33,653, U.S. Pat. Nos. 4,530,787, 4,569,790, 4,604,377, 4,748,234, 4,752,585, and 4,949,314. Pentafuside (FUZEON®) a 36-amino acid synthetic peptide that inhibits fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred.

In addition to the potential for CCR5 modulators in the management of HIV infections, the CCR5 receptor is an important regulator of immune function and compounds of the present invention may prove valuable in the treatment of disorders of the immune system. Treatment of solid organ transplant rejection, graft v. host disease, rheumatoid arthritis, inflammatory bowel disease, atopic dermatitis, psoriasis, asthma, allergies or multiple sclerosis by administering to a patient in need of such treatment an effective amount of a CCR5 antagonist compound of the present invention is also possible.

### Methods for Treating Rheumatoid Arthritis

Modulators of the CCR5 receptor may be useful in the treatment of various inflammatory conditions. Rheumatoid arthritis is characterized by infiltration of memory T lymphocytes and monocytes into inflamed joints. As leukocyte chemotactic factors, chemokines play an indispensable role in the attraction of macrophages to various tissues of the body, a process which is essential for both inflammation and the body's response to infection. Because chemokines and their receptors regulate trafficking and activation of leukocytes which contribute to the pathophysiology of inflammatory and infectious diseases, agents which modulate CCR5 activity, preferably antagonizing interactions of chemokines and their receptors, are useful in the therapeutic treatment of such inflammatory diseases.

Elevated levels of CC chemokines, especially CCL2, CCL3 and CCL5, have been found in the joints of patients with rheumatoid arthritis and have been correlated with the recruitment on monocytes and T cells into synovial tissues (I. F. Charo and R. M. Ransohoff, New Eng J. Med. 2006 354:610-621). T-cells recovered from synovial fluid of rheumatoid arthritis have been shown to express CCR5 and CXCR3. (P. Gao et al., J. Leukocyte Biol. 2003 73:273-280) Met-RANTES is an amino-terminal modified RANTES derivative which blocks RANTES binding to the CCR1 and CCR5 receptors with nanomolar potency. (A. E. Proudfoot et al., J. Biol. Chem. 1996 271:2599-2603). The severity of arthritis in rats adjuvant-induced arthritis was reduced by the administration of Met-RANTES. In addition, the levels of pro-inflammatory cytokines TNF-α and IL-1β were reduced. (S. Shahrara et al. Arthr. & Rheum. 2005 52:1907-1919) Met-RANTES has been shown to ameliorate the development of inflammation in an art recognized rodent model of inflammation, the collagen induced arthritis. (C. Plater-Zyberk et al. Immunol. Lett. 1997 57:117-120)

TAK-779 has also been shown to reduce both the incidence and severity of arthritis in the collagen-induced arthritis model. The antagonist inhibited the infiltration of inflammatory CCR5⁺ T-cells into the joint. (Y.-F. Yang et al., Eur. J. Immunol. 2002 32:2124-2132). Another CCR5 antagonist, SCH-X, was shown to reduce the incidence and severity of collagen-induced arthritis in rhesus monkeys. (M. P. M. Vierboom et al., Arthr. & Rheum. 2005 52(20):627-636).

In some anti-inflammatory conditions compounds of the present invention may be administered in combination with other anti-inflammatory drugs which may have a alternative mode of action. Compounds which may be combined with CCR5 antagonists include, but are not limited to:

(a) lipoxygenase antagonist or biosynthesis inhibitor such as an inhibitor of 5-lipoxygenase, (b) leukotriene antagonists (e.g., zafirlukast, montelukast, pranlukast, iralukast, pobilukast, SKB-106,203), (c) leukotriene biosynthesis inhibitors (e.g., zileuton, BAY-1005); (d) a non-steroidal antiinflammatory agent or cyclooxygenase (COX1 and/or COX2) inhibitor such as such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenarnic acid derivatives (flufenarnic acid, meclofenamic acid, mefenamic acid, niflumic acid and tolfenarnic acid), biphenylearboxylic acid derivatives (diflunisal and flufenisal), oxicarns (isoxicarn, piroxicam, sudoxicam and tenoxican), salicylates (acetyl salicylic acid, sulfasalazine), pyrazolones (apazone, bezpiperylon, feprazone, mofebutazone, oxyphenbutazone, phenylbutazone) and celecoxib; (e) a TNF inhibitor such as infliximab (REMICADE®), etanercept (ENBREL®), or adalimumab (HUMIRA®); (f) anti-inflammatory steroids such as beclomethasone, methylprednisolone, betamethasone, prednisone, dexamethasone, and hydrocortisone; (g) immunomodulators such as cyclosporine, leflunomide (ARAVA®), azathioprine (AZASAN®), penicillamine and levamisole; (h) folate antagonists such as methotrexate; (i) gold compounds such as aurothioglucose, gold sodium thiomalate or auranofin.

### Methods for Treating Transplant Rejection

Rejection following solid organ transplantation also is characterized by infiltration of T-cells and macrophages expressing the CCR5 receptor into the interstitial area. (J. Pattison et al., Lancet 1994 343:209-211) Renal transplant patients homozygous for the CCR5Δ32 deletion exhibit a significant survival advantage of patients heterozygous for the CCR5Δ32 deletion or homozygous wild type patients. (M. Fischerder et al., Lancet 2001 357:1758-1761) CCR5^{-/-} knock-out mice showed significant prolong graft survival in after transplantation of heart and islet tissue. (W. Gao et al., Transplantation 2001 72:1199-1205; R. Abdi et al., Diabetes 2002 51:2489-2495. Blocking the CCR5 receptor activation has been found to significantly extend cardiac allograph survival. (W. W. Hancock et al., Curr. Opin. Immunol. 2003 15:479-486).

In treatment of transplant rejection or graft vs. host diseases CCR5 antagonists of the present invention may be administered in combination with other immunosuppressive agents including, but are not limited to, cyclosporine (SANDIMMUNE®), tacrolimus (PROGRAF®, FK-506), sirolimus (RAPAMUNE®, rapamycin), mycophenolate mofetil (CELLCEPT®), methotrexate, anti-IL-2 receptor (anti-CD25) antibodies such as daclizumab (ZENAPAX®) or basiliximab (SIMULECT®), anti-CD3 antibodies visilizumab (NUVION®) or muromonab (OKT3, ORTHOCLONE®).

### Methods for Treating Asthma and COPD

Antagonism of the CCR5 receptor has been suggested as a target to inhibit of progression of asthma and COPD by antagonism of Th1 activation: B. Ma et al., J. Immunol. 2006 176(8):4968-4978, B. Ma et al., J. Clin. Investig. 2005 115(12):3460-3472 and J. K. L. Walker et al., Am. J. Respir. Cell Mo. Biol. 2006 34:711-718.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃ Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford).

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this application is based on AUTONOM™ v. 4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I

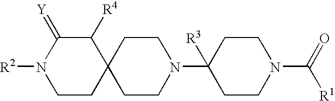

| Cpd. | | | | | | |
|---|---|---|---|---|---|---|
| # | R¹ | R² | R⁴ | Y | MS | mp |
| I-1 | 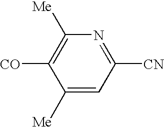 | 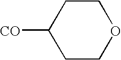 | n-Bu | H,H | 578 | |
| I-2 | 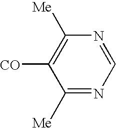 | 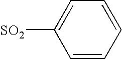 | n-Bu | H, H | 582 | |
| I-3 | 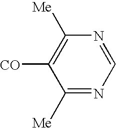 | 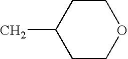 | n-Bu | O | 554 | |
| I-4 | 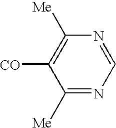 | 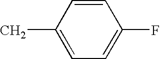 | n-Bu | O | 564 | |
| I-5 | 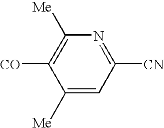 | 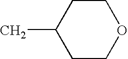 | n-Bu | O | 578 | |
| I-6 | 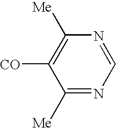 | 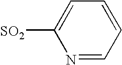 | n-Bu | H, H | 583 | |
| I-7 | 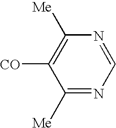 | 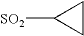 | n-Bu | H, H | 546 | |
| I-8 | 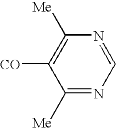 | 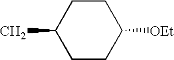 | n-Bu | O | 596 | |
| I-9 | 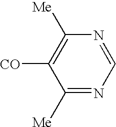 | SO₂Me | n-Bu | H, H | 520 | |
| I-10 | 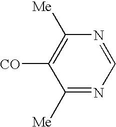 | 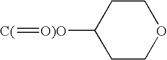 | n-Bu | H, H | 570 | |
| I-11 | 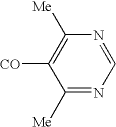 | SO₂NMe₂ | n-Bu | H, H | 549 | |
| I-12 | 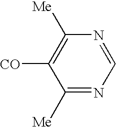 | 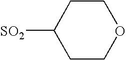 | n-Bu | H, H | 590 | |
| I-13 | 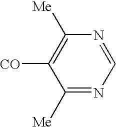 | 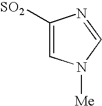 | n-Bu | H, H | 586 | |
| I-14 | 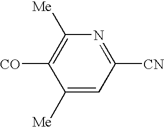 | 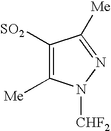 | n-Bu | H, H | 674 | |
| I-15 | 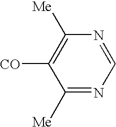 | 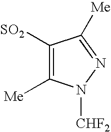 | n-Bu | H, H | 650 | |
| I-16 | 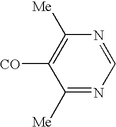 | 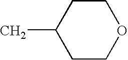 | n-Bu | H, H | 540 | |
| I-17 | 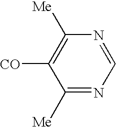 | 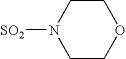 | n-Bu | H, H | 591 | |
| I-18 | 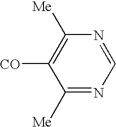 | 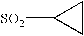 | OC₃H₇ | H, H | 548 | 99-100 |
| I-19 | 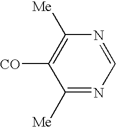 | 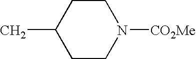 | n-Bu | O | 611 | |
| I-20 | 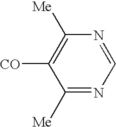 | 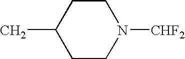 | n-Bu | O | 617 | |
| I-21 | 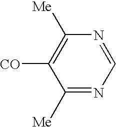 | 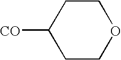 | n-Bu | H, H | 554 | |
| I-22 | 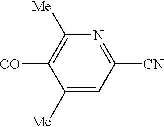 | SO₂NMe₂ | n-Bu | H, H | 573 | |
| I-23 | 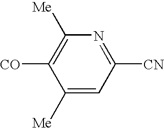 | 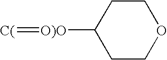 | n-Bu | H, H | 594 | |
| I-24 | 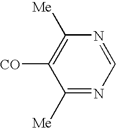 | 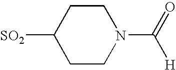 | n-Bu | H, H | 617 | 109.0-110 |
| I-25 | 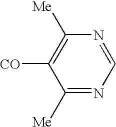 | 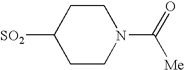 | n-Bu | H, H | 631 | 113.0-120 |
| I-26 | 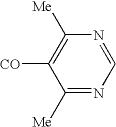 | SO₂Me | n-Bu | H, H | 519 | |
| I-27 | 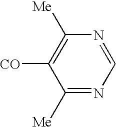 | 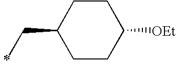 | n-Bu | O | 596 | |
| I-28 | 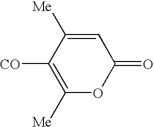 | 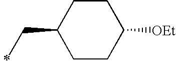 | n-Bu | O | 612 | |
| I-29 | 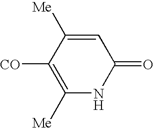 | 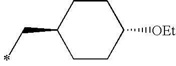 | n-Bu | O | 611 | |
| I-30 | 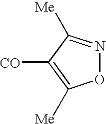 | 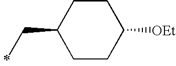 | n-Bu | O | 585 | |
| I-31 | 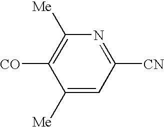 | 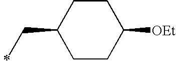 | n-Bu | O | 620 | |
| I-32 | 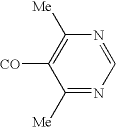 | 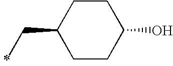 | n-Bu | O | 568 | |
| I-33 | 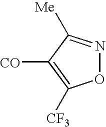 | 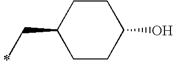 | n-Bu | O | 611 | |
| I-34 | 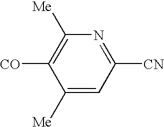 | 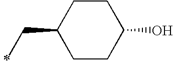 | n-Bu | O | 592 | |

Compounds of the present invention are prepared by condensation of 4-oxo-piperidine-1-carboxylic acid tert-butyl ester (A-6) with either 1-substituted-3,9-diaza-spiro[5.5]undecan-2-one

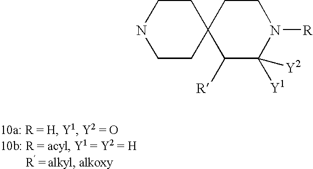

(10a) or 3-acyl-(1-substituted-3,9-diaza-spiro[5.5]undec-3-yl)-ethanone derivative (10b). In both 10a and 10b, the nitrogen occupying the 3-position is protected as an amide. The intermediate imine is sequentially contacted with Et₂AlCN and methyl magnesium bromide resulting in the introduction of the quaternary methyl substitutent. The N3 atom is functionalized by acylation, sulfonylation or alkylation. Cleavage of the Boc protecting group and acylation of the liberated secondary amine then affords compounds of the present invention.

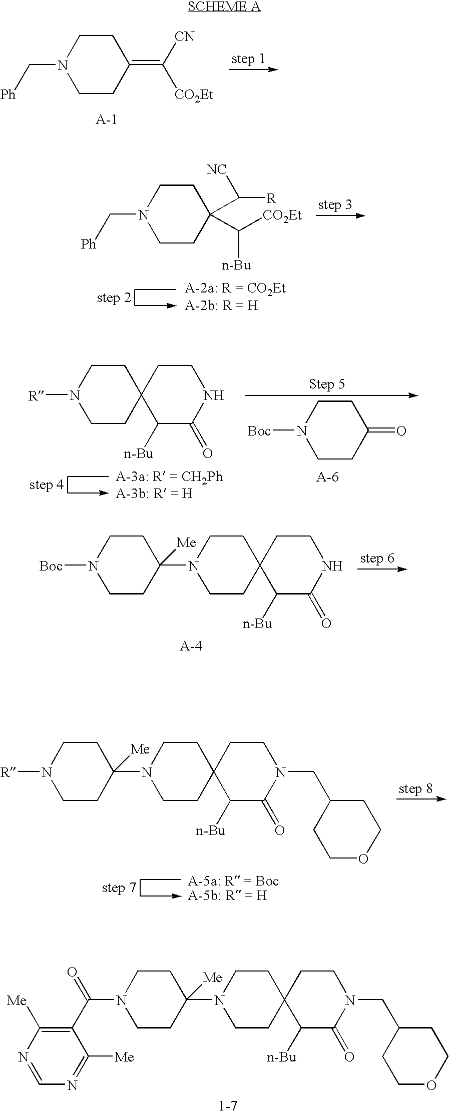

The synthesis of the requisite 3,9-diaza-spiro[5.5]undecan-2-one precursors of compounds of formula I wherein Y is O was accomplished as described in SCHEME A. Michael addition of an alkali metal enolate salt of an ester, e.g., ethyl hexanoate, to A-1 (CASRN 1463-52-1) afforded diester A-2b. Enolate salts of esters are prepared by treating the ester with a strong non-nucleophilic base. Commonly used bases include lithium dialkylamides, lithium hexamethyldisilazane, potassium or sodium tert-butoxide, and sodium or potassium hydride in inert solvents such as THF, dioxane, DME or DMF at temperature from −78° C. to RT, preferably from −78 to 0° C. The enolate thus formed can be contacted with an α,β-unsaturated carbonyl compound which results in 1,4-addition to the olefinic bond. Selective hydrolysis of the ester proximal to the nitrile with LiCl in aqueous DMSO (S. Mattsson et al. Tetrahedron Lett. 2007 48:2497-2499) afforded an acid which spontaneously decarboxylated to afford A-2b.

Reduction of the nitrile affords a primary amine which undergoes intra-molecular cyclization to afford the lactam A-3a. Reduction of a nitrile to an amine is well known and can be accomplished under known hydrogenation conditions in the presence of a metal catalyst, e.g. Raney nickel catalysts, palladium catalysts or platinum catalysts, preferably Raney nickel catalysts in an inert solvent, e.g. HOAc, alcohols, such as MeOH, EtOH; EtOAc, THF, and DMF. If desired, this reaction may be carried out in the presence or absence of an additive such as NH₄OH.

After cleavage of the benzyl protecting group from the N9 atom the 4-methyl-N-Boc-piperidine moiety was introduced by Ti(O-i-Pr)₄ mediated condensation of the secondary amine A-3b with N-Boc-4-oxopiperidine (A-6) and trapping the intermediate imine with Et₂AlCN which results in the introduction of a nitrile at the 4-position which is displaced with methyl magnesium bromide to afford A-4 (A. Palani et al. J. Med. Chem. 2001 44(21):3339-42).

Alkylation of the amide nitrogen was carried out under basic conditions by deprotonation of the amide proton and treating the resulting salt with an alkylating agent. Methods for alkylation of amides under basic conditions are well known in the art. The reaction is typically carried out in aprotic solvents such as THF, DMF, DMSO, NMP and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

Removal of the Boc protecting group and acylation or sulfonylation of the nitrogen affords the compounds of the present invention with an piperidone ring. Deprotection of the Boc group is carried out by well know methodology comprising acid treatment with TFA/DCM or HCl/dioxane.

Acylation of a amine can be effected by preparing an activated carboxylic acid into a more reactive form such as an acid chloride or a symmetrical or mixed acid anhydride and reacting the activated derivative with the amines of formula A-5b in a solvent such as DMF, DCM, THF, with or without water as a co-solvent, and the like at temperatures between 0° and 60° C. generally in the presence of a base such as Na₂CO₃, NaHCO₃, K₂CO₃, DIPEA, TEA or pyridine. Carboxylic acids are converted into their acid chlorides using standard reagents well known to someone skilled in the art, such as thionyl chloride, oxalyl chloride, phosphoryl chloride and the like. Those reagents can be used in presence of bases such as DIPEA, TEA or pyridine in inert solvent such as DCM or DMF.

Alternatively a carboxylic acid can be converted in situ into activated acids by different peptide coupling procedures known to those skilled in the art. These activated acids were reacted directly with the amines of formula A-5b to give the compounds of formula VI. Said activation with those peptide coupling procedures can involve the use of an activating agent like EDCI or DCC, HOBt, benzotriazol-1-yloxy-tris(dimethylamino)phosphonium hexafluorophosphate (BOP), bromo-tris-pyrrolidinophosphonium hexafluorophosphate (PyBrOP), or 2-fluoro-1-methylpyridinium p-toluenesulphonate (Mukaiyama's reagent) and the like with or without a base such NMM, TEA or DIPEA in an inert solvent such as DMF or DCM at temperatures between 0° C. and 60° C. The reaction may alternatively be carried out in presence of O-(7-azabenzotriazol-1-yl)-N,N,N′,N′-tetramethyluronium hexafluorophosphate (HATU) or 1-hydroxy-7-azabenzotriazole (HOAt) and TEA or DIPEA in DMF, DCM or THF. Acylation of amines (J. March, supra pp. 417-425; H. G. Benz, Synthesis of Amides and Related Compounds in Comprehensive Organic Synthesis, E. Winterfeldt, ed., vol. 6, Pergamon Press, Oxford 1991 pp. 381-411) has been reviewed.

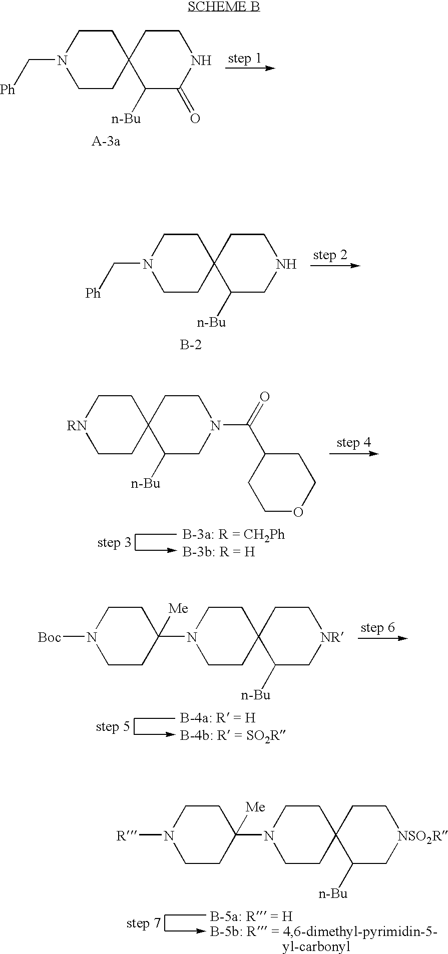

Compounds of the present invention according to formula I wherein Y is H,H (i.e., C<Y together is CH₂) were prepared by reduction of the piperidone and acylation of the resulting secondary amine prior to hydrogenolysis of the benzyl protecting group. Acylation is required to insure the formation of the imine in step 4 occurs at the correct position. Reduction of an amide is typically carried out with a hydride reducing agent such as LiAlH₄, BH₃-THF, DIBAL or sodium bis(2-methoxyethoxy)aluminumhydride in an inert etheral solvent. Acylation of the amine, hydrogenolysis of the benzyl protecting group and introduction of the piperidine moiety was carried out utilizing transformations describe above. During in the final step the reaction intermediate is treated with MeMgBr to introduce the quaternary methyl and concomitantly cleaves the tetrahydropyran-4-yl carbonyl protecting group to afford B-4a.

The secondary amine in B-4a can be further elaborated by acylation, sulfonylation of alkylation procedures. Acylations with acyl halides, activated carboxylic acid derivatives or alkoxy carbonyl chlorides are carried out by the general procedures described above. Sulfonylations are typically achieved by treating the amine with a sulfonyl chloride or sulfamoyl chloride in the presence of a base a tertiary amine base. Alkylation of amines are accomplished by treating the amine or a metal salt of the amine (i.e. a deprotonated form) with a alkylating agent RZ¹ wherein Z¹ is a leaving group such as halo, C₁₋₄ alkanesulphonyloxy, benzenesulphonyloxy or p-toluenesulphonyloxy, optionally in the presence of a base and/or a phase transfer catalyst such as 18-crown-6. The reaction may typically be carried out in the presence of a base such as TEA, DIPEA, DBU; or an inorganic base such as Na₂CO₃, NaHCO₃, K₂CO₃ or Cs₂CO₃: optionally in the presence of a phase transfer catalyst, and in a solvent such as MeCN, DMF, DMSO, 1,4-dioxane, THF or toluene. Alternatively, a metal salt of the amine (i.e. a deprotonated form) may be employed in a suitable solvent such as THF, DMF or 1,4-dioxane.

Removal of the Boc protecting group from B-4b and acylation of secondary amine B-5a is carried out as described in SCHEME A to afford the claimed compounds of formula I wherein Y is H,H.

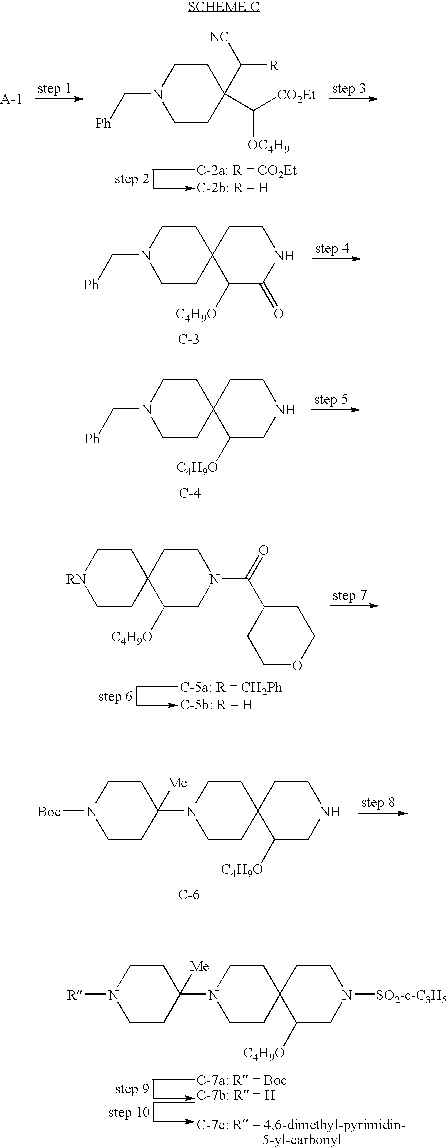

Compounds of the present invention according to formula I wherein R⁴ is C₁₋₆ alkoxy are prepared as described in SCHEMES A and B except in the initial Michael addition to A-1 the alkylating agent is an alkyl alkoxyacetate derivative as depicted in SCHEME C.

### Biological Assays

The capacity for novel compounds of the present invention to bind to the CCR5 receptor and thereby antagonize CCR5 function can be evaluated with assay systems known in the art (example 6). The capacity of compounds of the present invention to inhibit infection of CD4⁺/CCR5⁺ expressing cells can be determined using a cell-cell fusion assay as described in example 8 or an antiviral assay as described in example 9.

Functional assays directly measure the ability of a compound to produce a biologically relevant response or inhibit a response produced by a natural ligand (i.e., characterizes the agonist vs. antagonist properties of the test compounds). In a calcium flux assay, cells expressing the CCR5 are loaded with calcium sensitive dyes prior to addition of compound or the natural CCR5 ligand. Compounds with agonist properties will induce a calcium flux signal in the cell, while the compounds of this invention are identified as compounds which do not induce signaling by themselves but are capable of blocking signaling by the natural ligand RANTES.

### Dosage and Administration

The compounds of the present invention may be formulated in a wide variety of oral administration dosage forms and carriers. Oral administration can be in the form of tablets, coated tablets, dragées, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w).

The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### EXAMPLE 1

### 1-Butyl-9-[1-(4,6-dimethyl-pyrimidine-5-carbonyl)-4-methyl-piperidin-4-yl]-3-(tetrahydro-pyran-4-ylmethyl)-3,9-diaza-spiro[5.5]undecan-2-one (I-3, SCHEME A)

Step 1—To a solution of diisopropylamine (4.88 mL, 34.9 mmol) in THF (100 mL) cooled to −78° C. was added n-BuLi (2.5 M in hexane, 13.3 mL, 33.3 mmol) and the reaction was stirred for 15 min. The dry-ice acetone bath was removed and stirring was continued for another 20 min then the reaction mixture was re-cooled to −78° C. To the LDA solution was added dropwise via a syringe over 10 min a solution of ethyl caproate (5.5 mL, 33.3 mmol) in THF (30 mL) which was pre-cooled to −78° C. The reaction was stirred at −78° C. for 40 min. A solution of A-1 (8.6 g, 30 mmol) in THF (30 mL) was added via a syringe. The reaction mixture was poured into a mixture of H₂O and EtOAc. The organic layer was separated and the aqueous layer was extracted with EtOAc. The combined organic extracts were dried (MgSO₄), filtered, and concentrated. The residue was purified by SiO₂ column chromatography eluting with an EtOAc/hexane gradient (20% to 40% EtOAc over 30 min) to afford 11.71 g (90%) of a diastereomeric mixture of A-2a as an oil.

¹HNMR (CDCl₃, 300 MHz) δ 7.32-7.24 (m), 4.30-4.08 (m), 3.51 (s), 2.81-2.26 (m), 2.15-1.95 (m), 1.90-1.65 (m), 1.40-1.15 (m), 0.91-0.85 (m); IR (neat film) 3062, 3027, 2958, 2873, 2810, 2769, 2246, 1736, 1604, 1495, 1454, 1370, 1320, 1249, 1181, 1074, 1030, 857, 740, 699 cm⁻¹; MS calcd for C₁₅H₃₇N₂O₄ [M+H]⁺ 429. Found, 429.

Step 2—A suspension of A-2a (15.46 g, 36.1 mmol) and LiCl (3.06 g, 72.2 mmol) in DMSO (100 mL) and H₂O (10 mL) was heated at 200° C. for 1.5 h. After cooling to RT, the content was diluted with EtOAc and the solution washed with 50% aqueous saturated brine. The organic layer was separated. The aqueous layer was twice extracted with EtOAc. The combined extracts were dried (MgSO₄), filtered, and concentrated. The residue was purified by SiO₂ chromatography eluting with an EtOAc/hexane gradient (10% to 50% EtOAc over 36 min) to give 11.5 g of A-2b as an oil.

Elemental Analysis Calcd for C₁₉H₂₅N₂O₂: C, 74.12%; H, 9.05; N, 7.86. Found: C, 73.71; H, 8.89; N, 7.74.

Step 3—A mixture of A-2b (3.9 g, 11 mmol) and Raney-Nickel (a slurry in water, ˜5 g) in MeOH (25 mL) was shaken under 55 psi of H₂ atmosphere overnight. The reaction mixture was filtered through a plug of CELITE® and concentrated to afford a viscous oil. The oil was dissolved in toluene (15 mL) and heated at reflux for 2 h. The reaction mixture was purified by SiO₂ chromatography eluting with DCM/DCM:MeOH:28% aqueous NH₄OH (60:10:1) gradient (60 to 30% DCM over 30 min) to afford 1.62 g of A-3a as a viscous oil: MS calcd for C₂₀H₃₀N₂O [M+H]⁺ 315. Found: 315.

Step 4—A mixture of A-3a (0.83 g, 2.64 mmol) and 20% Pd(OH)₂/C in EtOH was shaken under 60 psi of H₂ atmosphere overnight. The content was filtered through a plug of CELITE® and concentrated under in vacuo to afford 0.74 g of crude A-3b as an oil, which was used in the next step without further purification: MS calcd for C₁₃H₂₄N₂O [M+H]⁺ 225. Found: 225.

Step 5—To a solution of A-3b (0.74 g) and N-Boc-4-piperidone (0.725 g, 3.64 mmol) in DCE under argon was added Ti(O-i-Pr)₄ (1.26 mL, 4.3 mmol). The reaction was stirred at RT overnight then Et₂AlCN (1.0 M in toluene, 5 mL, 5 mmol) was added dropwise. After stirring at RT for 5 h, the reaction was quenched with saturated aqueous NaHCO₃ (ca. 0.5 mL). The content was vigorously stirred at RT for 30 min, filtered through a plug of CELITE® and the solvent evaporated to afford 1.07 g of A-4 as a pale yellow foam. The foam was dissolved in anhydrous THF (15 mL) and cooled with a dry ice/acetone bath. A solution of MeMgBr (3 M in Et₂O, 7.7 mL, 23.1 mmol) was added drop-wise. The bath was removed and the reaction was stirred at RT overnight. The content was cooled with an ice bath and quenched with saturated aqueous NH₄Cl. The mixture was made basic with 28% aqueous NH₄OH and extracted with EtOAc. The combined organic layer was dried (MgSO₄), filtered and concentrated. The residue was purified by SiO₂ chromatography eluting with DCM/DCM:MeOH:28% aqueous NH₄OH (60:10:1) gradient (80% to 30% DCM over 25 min) to afford 0.517 g of A-4 as a white foam: MS calcd for C₂₀H₃₀N₂O [M+H]⁺ 315. Found: 315.

Step 6—To a suspension of A-4 (0.30 g, 0.71 mmol) in DMF (5 mL) at RT was added NaH (60% in mineral oil, 0.143 g, 3.56 mmol). The reaction was stirred at RT for 30 min then neat 4-bromomethyl-tetrahydropyran (0.2 mL) was added. The reaction was warmed in a 50° C. bath and stirred for 1 h. Additional 4-bromomethyl-tetrahydropyran (0.29 mL) was added over a 3 h period. The reaction was stirred at 50° C. overnight. More NaH (30 mg, 60% in mineral oil) and 4-bromomethyl tetrahydropyran were added. Stirring was continued at 50° C. for one additional hour, cooled to RT, and quenched with ice. The mixture was diluted with 50% saturated brine and extracted with EtOAc. The combined extracts were dried (Na₂SO₄), filtered, and concentrated. The residue was purified by SiO₂ chromatography eluting with DCM/DCM:MeOH:28% aqueous NH₄OH (60:10:1) gradient (80% to 30% DCM over 45 min) to afford 0.27 g of A-5a as a white foam: MS calcd for C₃₀H₅₃N₃O₄ [M+H]⁺ 520. Found: 520.

Steps 7 & 8—To a solution of A-5a (0.27 g, 0.52 mmol) in DCM (2 mL) at RT was added TFA (0.4 mL). The reaction was stirred at RT for 45 min, quenched with ice-cold saturated aqueous NaHCO₃, and extracted with EtOAc. The aqueous layer was concentrated in vacuo. The residual solid was washed with EtOAc. EtOAc extracts were combined, dried (MgSO₄), filtered, and concentrated to afford 114 mg of A-5b as a pale yellow oil, which was used in the next step without further purification: MS calcd for C₂₅H₄₆N₃O₂ [M+H]⁺ 420. Found: 420.

To a mixture of A-5b (114 mg, assuming 100% purity, 0.27 mmol), 4,6-dimethyl-pyrimidine-5-carboxylic acid (54 mg, 0.35 mmol), EDCI (142 mg, 0.74 mmol), HOBt (73 mg, 0.54 mmol) at RT were added sequentially DCM (7 mL) and DIPEA (0.47 mL, 2.7 mmol). The mixture was stirred at RT overnight, quenched with saturated aqueous NaHCO₃, and extracted with EtOAc. The combined extracts were dried (Na₂SO₄), filtered, and concentrated in vacuo. The residue was purified by SiO₂ chromatography eluting with DCM/DCM:MeOH:28% aqueous NH₄OH (60:10:1) gradient (90% to 25% DCM over 45 min) to afford 0.075 g of 1-3 as a white foam: MS calcd for C₃₂H₅₂N₅O₃ [M+H]⁺ 554. Found, 554.

Compound I-5 was made analogously except in step 8, 4,6-dimethyl-pyrimidine-5-carboxylic acid was replaced with 6-cyano-2,4-dimethyl-nicotinic acid.

Compounds I-8 and I-4 were prepared analogously except in step 6, 4-bromomethyl-tetrahydropyran was replaced with trans-4-ethoxy-cyclohexanol p-toluenesulfonate ester 4-fluoro-benzylbromide.

### EXAMPLE 2

### 4-{1-Butyl-9-[1-(4,6-dimethyl-pyrimidine-5-carbonyl)-4-methyl-piperidin-4-yl]-2-oxo-3,9-diaza-spiro[5.5]undec-3-ylmethyl}-piperidine-1-carboxylic acid methyl ester (I-19)

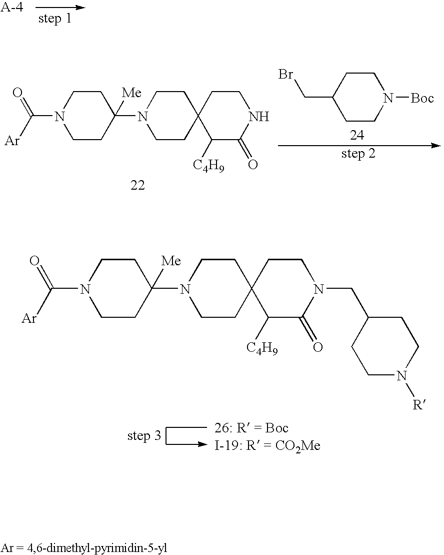

Step 1—The amine A-4 was converted to 22 by the procedure described for steps 7 and 8 of example 1 except A-4 was the starting material rather than A-5a and afforded 1.032 g of 22 as a white foam: MS calcd for C₂₃H₃₅N₅O₂ [M+H]⁺ 456. Found: 456.

Step 2—To a suspension of the 22 (0.711 g, 1.56 mmol) in THF (15 mL) at RT was added NaH (60% in mineral oil, 0.075 g, 1.87 mmol). The reaction was heated at reflux for 1 h, cooled to RT and neat 4-bromomethyl-piperidine-1-carboxylic acid tert-butyl ester (24, 0.87 g, 3.12 mmol) was added. The reaction was heated at reflux for 72 h. The reaction was diluted with DCM, filtered through CELITE® and concentrated in vacuo. The residue was purified by SiO₂ chromatography eluting a with DCM/DCM:MeOH:28% aqueous NH₄OH (60:10:1) gradient (70% to 30% DCM over 45 min) to afford 0.219 g of 26 as a white foam: MS calcd for C₃₇H₆₀N₆O₄ [M+H]⁺ 653. Found: 653.

Step 3—The Boc-protecting group on 26 was removed as described in step 7 of example 1. To the corresponding amine (0.09 g, 0.162 mmol) in DCM (10 mL) at RT was added sequentially methyl chloroformate (0.019 mL, 0.244 mmol) and DIPEA (0.056 mL, 0.325 mmol). The reaction was stirred at RT for 90 min, quenched with saturated aqueous NaHCO₃ and product extracted with DCM (5×20 mL). The combined extracts were dried (MgSO₄) and concentrated. The residue was purified by preparative thin layer chromatography developed with 40/60 solution of DCM/DCM:MeOH:28% aqueous NH₄OH (60:10:1) to afford 81 mg I-19 as a white foam: MS calcd for C₃₄H₅₄N₆O₄ [M+H]⁺ 611. Found: 611.

I-20 was prepared analogously except is step 3, the acylation with methyl chloroformate was omitted and the piperidine nitrogen was alkylated in a solution containing 2,2-difluoroethyl trifluoromethanesulfonate, DIPEA and MeCN.

On skilled in the art will appreciate that the piperidine 26 (R′=H) can be further transformed into compounds within the scope of the present invention by acylation, sulfonylation or alkylation with a variety of carboxylic acids (or derivatives thereof), sulfonyl chlorides or alkylating agents which are readily available.

### EXAMPLE 3

### {4-[7-Butyl-9-(tetrahydro-pyran-4-sulfonyl)-3,9-diaza-spiro[5.5]undec-3-yl]-4-methyl-piperidin-1-yl}-(4,6-dimethylpyrimidin-5-yl)-methanone (I-12; SCHEME B, R″=4-tetrahydropyran-4-yl)

Step 1—To a solution of A-3a (0.53 g, 1.7 mmol) in THF (10 mL) at RT was added LiAlH₄ (1M in THF, 3.5 mL, 3.5 mmol). The reaction was heated at reflux for 4 h, cooled to RT, and quenched sequentially with H₂O (0.35 mL), 1N aqueous NaOH (0.7 mL) and H₂O (0.8 mL). The mixture was stirred vigorously for 1 h at 0° C., diluted with MeOH, and filtered through a pad of CELITE®. The filtrate was concentrated to afford ca. 0.5 g of B-2 as a viscous oil, which was used in the next step without further purification: MS calcd for C₂₀H₃₃N₂ [M+H]⁺ 301. Found: 301.

Step 2—To a mixture of B-2 from step 1 (500 mg, assuming 100% purity, 1.67 mmol), tetrahydropyran-4-carboxylic acid (228 mg, 1.75 mmol), EDCI (449 mg, 2.32 mmol), HOBt (284 mg, 2.1 mmol) and DCM (7 mL) at RT was added DIPEA (0.47 mL, 2.7 mmol). The mixture was stirred at RT overnight, quenched with saturated aqueous NaHCO₃, and extracted with DCM. The combined extracts were dried (MgSO₄), filtered and concentrated. The residue was purified by SiO₂ chromatography eluting with DCM/DCM:MeOH:28% aqueous NH₄OH (60:10:1) gradient (75 to 50% DCM over 22 min, then 50 to 40% DCM over 8 min) to afford 0.494 g of B-3a: MS calcd for C₂₆H₄₁N₂O₂ [M+H]⁺ 413. Found: 413.

Step 3—A suspension of B-3a (0.18 g, 0.44 mmol) and 20% Pd(OH)₂/C (0.1 g) in EtOH (15 mL) was shaken under 60 psi of H₂ overnight. The suspension was filtered through a plug of CELITE® and concentrated in vacuo to afford B-3b, which was used in the next step without further purification: MS calcd for C₁₉H₃₆N₂O₂ [M+H]⁺ 323. Found: 323.

Step 4—To a solution of B-3b (assuming 0.44 mmol) and N-Boc-4-piperidone (92 mg, 0.46 mmol) in anhydrous DCE (3 mL) at RT was added Ti(Oi-Pr)₄ (0.18 mL, 0.62 mmol). After stirring at RT overnight, a solution of Et₂AlCN (0.7 mL, 1M in toluene, 0.7 mmol) was added and stirring continued overnight. The reaction was quenched with saturated aqueous NaHCO₃ (1 mL). The solution was vigorously stirred at RT for 20 min and filtered through a plug of CELITE®. The filter cake was rinsed with DCM. The combined filtrate was washed with H₂O and brine, dried (Na₂SO₄), filtered and concentrated. The residue was dissolved in THF (2 mL) at RT and MeMgBr (3M in Et₂O, 1 mL) was added. The reaction was stirred at RT for 4 d, cooled to 0° C. and quenched with saturated aqueous NH₄Cl. The mixture was made basic to pH 9 with 28% aqueous NH₄OH and thrice extracted with EtOAc. The combined extracts were dried (Na₂SO₄), filtered, and concentrated. The residue was purified by SiO₂ chromatography eluting with a DCM/DCM:MeOH:28% aqueous NH₄OH (60:10:1) gradient (70 to 15% DCM over 20 min) to afford 0.068 g of B-4a: MS calcd for C₂₄H₄₆N₃O₂ [M+H]⁺ 408. Found: 408.

Step 5—To a solution of B-4a (200 mg, 0.5 mmol) and TEA (0.28 mL, 2 mmol) in DCM (4 mL) at RT was added tetrahydropyran-4-sulfonyl chloride (144 mg, 1 mmol). The reaction was stirred at RT for 45 min, quenched with saturated aqueous NaHCO₃, and extracted with DCM. The combined extracts were dried (Na₂SO₄), filtered, and concentrated. The residue was purified by SiO₂ chromatography eluting with a DCM/DCM:MeOH:28% aqueous NH₄OH (60:10:1) gradient (90 to 50% DCM over 20 min) to afford 0.230 g of B-4b as a foam: MS calcd for C₂₉H₅₄N₃O₅S [M+H]⁺ 556. Found: 556.

Step 6—To a solution of B-4b in DCM (4 mL) at RT was added TFA (0.8 mL). The reaction was stirred at RT for 1.5 h, quenched with saturated aqueous NaHCO₃, and extracted with CHCl₃. The combined extracts were dried (MgSO₄), filtered and concentrated to afford 0.2 g of crude B-5a as a yellow foam, which was used in the next step without further purification.

Step 7—To a mixture of the B-5a from step 6,4,6-dimethyl-pyrimidine-5-carboxylic acid (134 mg, 0.88 mmol), EDCI (192 mg, 1.0 mmol), HOBt (135 mg, 1.0 mmol) and DCM (5 mL) under argon at RT was added DIPEA (0.7 mL, 4 mmol). The reaction was stirred at RT overnight, quenched with 1/1 saturated aqueous NaHCO₃/H₂O, and extracted with CHCl₃. The combined extracts were dried (MgSO₄), filtered and concentrated. The residue was purified by SiO₂ chromatography eluting with a DCM/DCM:MeOH:28% aqueous NH₄OH (60:10:1) gradient (80 to 20% DCM over 30 min) to afford 0.190 g of product which was further purified by preparative TLC to afford 0.070 g of 1-12 (B-5b, R″=tetrahydropyran-4-yl) which was >99% pure as a white foam. MS calcd for C₃₁H₅₂N₅O₄S [M+H]⁺ 590. Found: 590.

I-2, I-6, I-7, I-9, I-13, and I-15 were prepared analogously except in step 5, the following sulfonyl chlorides were used in place of tetrahydropyran-4-sulfonyl chloride: benzenesulfonyl chloride, 2-pyridinesulfonyl chloride, cyclopropanesulfonyl chloride, methanesulfonyl chloride, 1-methylimidazole-4-sulfonyl chloride (CASRN 137049-00-4) and 1-difluoromethyl-3,5-dimethyl-1H-pyrazole-4-sulfonyl chloride (CASRN 943152-92-9). The sulfamides I-11 and I-17 were prepared analogously except in step 5, tetrahydropyran-4-sulfonyl chloride was replaced with N,N-dimethylsulfamoyl chloride and 4-morpholinesulfonyl chloride, respectively.

I-14 and I-22 were prepared analogously using 1-difluoromethyl-3,5-dimethyl-1H-pyrazole-4-sulfonyl chloride and N,N-dimethylsulfamoyl chloride in place of tetrahydropyran-4-sulfonyl chloride in step 5 and 2-4,dimethyl-6-cyano-pyridine-3-carboxylic acid in place of 4,6-dimethyl-pyrimidine-5-carboxylic acid in step 7.

### EXAMPLE 4

### 1-Butyl-9-[1-(4,6-dimethyl-pyrimidine-5-carbonyl)-4-methyl-piperidin-4-yl]-3,9-diaza-spiro[5.5]undecane-3-carboxylic acid tetrahydro-pyran-4-yl ester (I-10)

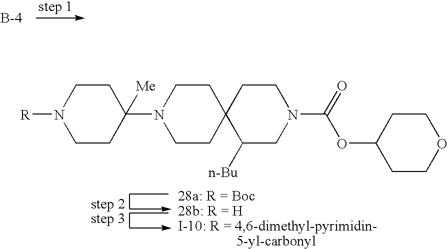

Step 1—To a suspension of disuccinimidyl carbonate (0.165 g, 0.65 mmol) and 4-hydroxytetrahydropyan (0.04 mL, 0.43 mmol) in MeCN (3 mL) at RT was added TEA (0.18 mL, 1.3 mmol). After stirring overnight the reaction was a clear solution. The solution was added to a suspension of B-4 (0.145 g, 0.36 mmol) in MeCN (3 mL). After stirring at RT for 4 h, the content was diluted with half saturated aqueous NaHCO₃ and extracted with CHCl₃. The combined extracts were dried (Na₂SO₄), filtered, and concentrated. The residue was purified by SiO₂ chromatography eluting with a DCM/DCM:MeOH:28% aqueous NH₄OH (60:10:1) gradient (80 to 65% DCM over 25 min) to afford 0.142 g of 28a as an off-white foam: MS calcd for C₃₀H₅₄N₃O₅ [M+H]⁺ 536. Found: 536.

Step 2—A suspension of 28a (140 mg, 0.26 mmol) in 3 M HCl in 1,4-dioxane (7 mL) was stirred at RT for 2 h. The content was concentrated under reduced pressure to afford 128 mg of 28b as a crude oil which was used in the next step without further purification: MS calcd for C₂₆H₄₆N₃O₃ [M+H]⁺ 436. Found: 436.

Step 3—A solution of 28b (60 mg), 4,6-dimethyl-pyrimidine-5-carboxylic acid (26 mg, 0.17 mmol), EDCI (40 mg, 0.21 mmol), HOBt (28 mg, 0.21 mmol), and DIPEA (1.9 mL, 1.1 mmol) and SCM (3 mL) were treated as described in step 7 of example 3 to afford 0.018 g of I-10: MS calcd for C₃₂H₅₂N₅O₄ [M+H]⁺ 570. Found: 570.

### EXAMPLE 5

### {1-Butyl-9-[1-(4,6-dimethyl-pyrimidine-5-carbonyl)-4-methyl-piperidin-4-yl]-3,9-diaza-spiro[5.5]undec-3-yl}-(tetrahydro-pyran-4-yl)-methanone (I-21)

To a solution of B-4a (175 mg, 0.43 mmol), tetrahydropyran-4-carboxylic acid (61 mg, 0.47 mmol), EDCI (142 mg, 0.54 mmol), HOBt (73 mg, 0.54 mmol), and DIPEA (0.38 mL, 2.2 mmol) in DCM (5 mL) under argon at room temperature. The crude product was purified by SiO₂ chromatography eluting with a DCM/DCM:MeOH:28% aqueous NH₄OH (60:10:1) gradient (90 to 50% DCM over 20 min) to afford to gave 76 mg of B-4b (R′=tetrahydropyran-4-yl-carbonyl) as a foam: MS calcd for C₃₀H₅₄N₃O₄ [M+H]⁺ 520. Found, 520.

The compound from the previous step was converted to I-21 as described in steps 2 and 3 of example 4. I-1 was prepared analogously except in the final step 2,4-dimethyl-6-cyano-nicotinic acid was used in place of 4,6-dimethyl-pyrimidine-5-carboxylic acid.

### EXAMPLE 6

### [4-(9-Cyclopropanesulfonyl-7-propoxy-3,9-diaza-spiro[5.5]undec-3-yl)-4-methyl-piperidin-1-yl]-(4,6-dimethyl-pyrimidin-5-yl)-methanone (I-18)

Preparation of Ethyl Propoxyacetate

To a mixture of n-propanol (50 mL, 670 mmol) and rhodium octanoate dimer (0.5 g, 0.64 mmol) at RT was added ethyl diazoacetate (20 g, 175 mmol) via a syringe pump over 3 h. The reaction was stirred at RT overnight. Distillation under reduced pressure (ca. 25 mm Hg, 78-83° C.) afforded 18.07 g of the title compound as a clear oil: ¹H NMR (300 MHz, CDCl₃): δ 4.22 (q, J=7.1 Hz, 2H), 4.07 (s, 2H), 3.49 (t, J=6.7 Hz, 2H), 1.71-1.59 (m, 2H), 1.29 (t, J=7.2 Hz, 3H), 0.95 (t, J=7.4 Hz, 3H).

Step 1—The condensation was carried out using ethyl propoxyacetate (4.46 g, 31.0 mmol), LDA (1M in THF, 30.9 mL, 30.9 mmol), and A-1 (5.5 g, 19.3 mmol) using the procedure described in step 1 of example 1. The crude product was purified by SiO₂ eluting with an EtOAc/hexane gradient (20-40% EtOAc over 40 min) to afford 6.2 g of C-2a: MS calcd for C₂₄H₃₅N₂O₅ [M+H]⁺ 431. Found, 431.

Step 2—A solution of C-2a (6.2 g, 1.4 mmol) and LiCl (1.22 g, 28.8 mmol) in a mixture of DMSO (43 mL) and H₂O (4.3 mL) were allowed to reaction as described in step 2 of example 1. The crude product was purified by SiO₂ chromatography eluting with an EtOAc/hexane gradient (30 to 60% EtOAc) to afford C-2b: MS calcd for C₂₁H₃₁N₂O₃ [M+H]⁺ 359. Found: 359.

Step 3—To a solution of C-2b (4.37 g, 12.2 mmol) and CoCl₂ hexahydrate (6.39 g, 26.9 mmol) in MeOH (60 mL) at RT was added NaBH₄ (6.9 g, 183 mmol) portion-wise. After stirring at RT overnight, the content was concentrated under reduced pressure, diluted with a mixture of CHCl₃ and saturated aqueous NH₄OH, and filtered through a pad of CELITE®. The filtrate was transferred to a separatory funnel. The organic layer was separated and the aqueous layer was extracted with CHCl₃. The combined extracts were concentrated in vacuo. The residue was purified by SiO₂ chromatography eluting with a DCM/DCM:MeOH:28% aqueous NH₄OH (60:10:1) gradient (70 to 30% DCM over 40 min) to afford 3 g of C-3 as a yellow oil: MS calcd for C₁₉H₂₉N₂O₂ [M+H]⁺ 317. Found, 317.

Step 4—The reduction of a solution of C-3 (2.96 g, 9.4 mmol) and THF (100 mL) using LiAlH₄ (1M in THF, 30 mL, 30 mmol). was carried out as described in step 1 of example 3 to afford 2.11 g of D-4 as a viscous oil: MS calcd for C₁₉H₃₁N₂O [M+H]⁺ 303. Found, 303.2.

Step 5—A mixture of C-4 (2.11 g, 7 mmol), tetrahydropyran-4-carboxylic acid (1.0 g, 7.7 mmol), EDCI (1.74 g, 9.1 mmol), HOBt (1.23 g, 9.1 mmol), DIPEA (6.1 mL, 35 mmol) and DCM were reacted as described in step 2 of example 3. The crude viscous oil was purified by SiO2 chromatography eluting with a DCM/DCM:MeOH:28% aqueous NH₄OH (60:10:1) gradient (75 to 35% DCM over 30 min) to afford C-5a: MS calcd for C₂₅H₃₉N₂O₃ [M+H]⁺ 415. Found, 415.

Step 6—A mixture of C-5a (2.42 g) and 20% Pd(OH)₂/C (1 g) was shaken under 55 psi of H₂ as described in step 3 of example 3 to afford C-5b as a viscous oil which was used without further purification: MS calcd for C₁₈H₃₃N₂O₃ [M+H]⁺ 325. Found, 325.

Step 7—The reductive amination and methylation of C-5b (1.93 g) and N-Boc-4-piperidone was carried out as described in step 4 of example 3. The crude product was purified by SiO₂ chromatography eluting with a DCM/DCM:MeOH:28% aqueous NH₄OH (60:10:1) gradient (70 to 12% DCM over 45 min) to afford 0.297 g of C-6 as a white foam: MS calcd for C₂₃H₄₄N₃O₃ [M+H]⁺ 410. Found, 410.

Step 8—A solution of C-6 (297 mg, 0.73 mmol), cyclopropanesulfonyl chloride (124 mg, 0.88 mmol), and TEA (0.34 mL, 2.5 mmol) in DCM (6 mL) were reacted as described in step 5 of example 3. The crude product was purified by SiO₂ chromatography eluting with a DCM/DCM:MeOH:28% aqueous NH₄OH (60:10:1) gradient (70 to 35% DCM over 20 min) to afford 0.38 g of C-7a as a pale-yellow foam: MS calcd for C₂₆H₄₈N₃O₅S [M+H]⁺ 514. Found, 514.

Steps 9 & 10—A sample of 0.38 g of C-7a was converted to 1-18 as described in steps 6 & 7 of example 3. The crude product was purified by SiO₂ chromatography eluting with a DCM/DCM:MeOH:28% aqueous NH₄OH (60:10:1) gradient (70 to 25% DCM over 40 min) to afford 0.247 g of 1-18 as a pale-yellow foam: HRMS calcd for C₂₈H₄₆N₅O₄S [M+H]⁺ 548.3271. Found, 548.3290; mp 99-100° C.

### REFERENTIAL EXAMPLE 7

### 1-Benzyloxy-3,5-dimethyl-1H-pyrazole-4-carboxylic acid (34b)

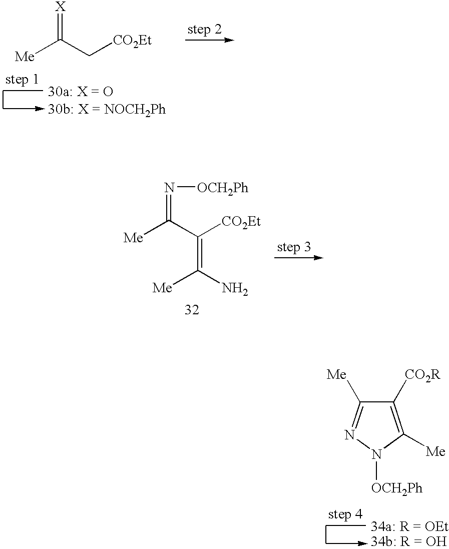

Step 1—O-benzyl-hydroxylamine hydrochloride (14.2 g) was suspended in saturated NaHCO₃ and the insoluble solid extracted with benzene and the solution dried (MgSO₄), filtered and the filtrate combined with ethyl acetoacetate (15 g) and MgSO₄. The resulting reaction mixture was stirred at RT overnight. The MgSO₄ was filtered and the solution concentrated in vacuo. The crude product was purified by SiO₂ chromatography eluting with 20% EtOAc/hexane to afford 12 g of 30b.

Step 2—To a solution of 30b (10 g, 42.5 mmol) in MECN (80 mL) cooled to 0° C. A solution of SnCl₄ in DCM (42.5 mL, 1M DCM solution) was added through an addition funnel at a rate that maintained internal temperature at 3-4° C. (internal temperature. The reaction mixture was stirred 0° C. for 1 h. Maintenance of low temperatures during the workup is critical. The reaction mixture was poured onto ice while cooling the ice phase in an external ice bath. The reaction was quenched by addition of NaHCO₃ solution followed by solid NaHCO₃ until the solution was basic. The NaHCO₃ was added very slowly (ca 5 h) while maintaining the internal temperature at 0° C. The solution was diluted with EtOAc and filtered through CELITE®. The organic phase was washed with brine, dried (MgSO₄), filtered and evaporated. The crude product was purified by SiO₂ chromatography eluting with 5% MeOH/DCM to afford 9.7 g of 32.

Step 3—A solution of 32 (1 g, 3.64 mmol), Cu(OAc)₂ (0.926 g, 5.1 mmol) and pyridine (10 mL) was stirred at 100° C. for 6 h. The reaction was cooled to RT and made basic with 10% NH₄OH and filtered through CELITE®. The filtrate was extracted with EtOAc, dried (MgSO₄), filtered and evaporated. The crude product was purified by SiO₂ chromatography eluting with 50% EtOAc/hexane to afford 340 mg of 34a.

Step 4—A solution of 34a (0.340 g, 1.23 mmol), 1N aqueous NaOH (5 mL), EtOH (5 mL) and dioxane (10 mL) was heated at 75° C. for 2 h. The reaction mixture was cooled to RT, concentrated and made acidic (pH ca. 2-3) with 1N HCl. The solvent was evaporated and the residue triturated with EtOAc. The solid was removed by filtration and the solvent evaporated to afford 0.286 g of 34b which was dried under a high vacuum.

The acid 34b was incorporated onto the piperidine scaffold using EDCI/HOBt/DIPEA condensation as described previously. Modification of the N-oxide substituent could be carried out after coupling to the piperidine. Thus hydrogenolysis (Pd/Ba₂SO₄, H₂, MeOH) afforded the corresponding N-oxide which, in turn, could be alkylated (NaH, Me₃SiCN₂, THF)

### 5-Cyano-2,4-dimethyl-1H-pyrrole-3-carboxylic acid (38)

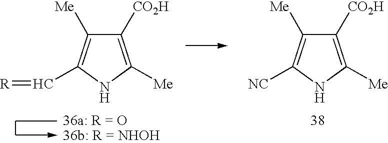

A slurry of 36a (10 g, 35.2 mmol, CASRN 253870-02-9) in MeOH and EtOH (ca. 1:1) was added to NaHCO₃ (5 g) and hydroxylamine hydrochloride (4.1 g) dissolved in a minimal amount of water. The reaction mixture was stirred overnight at RT. The solvents were evaporated and purified by SiO2 chromatography eluting with 5% MeOH/CHCl₃ to afford 36b.

A solution of the 36b (0.250 g) and acetic anhydride (5 mL) were heated at efflux overnight. The reaction mixture was cooled and the solvent evaporated. The crude product which was N-acetylated. The N-acylpyrrole (1 g) was dissolved in MeOH (30 mL) and 1N NaOH (5 mL) was added and the reaction The reaction mixture was heated at reflux for three days. The reaction mixture was cooled and the solvents were evaporated. The residue was dissolved in H₂O (5 mL) and acidified with 1N HCl and extracted with EtOAc. The organic extract was dried (MgSO₄), filtered and evaporated to afford 0.800 g of 38.

### EXAMPLE 8

### 1-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one

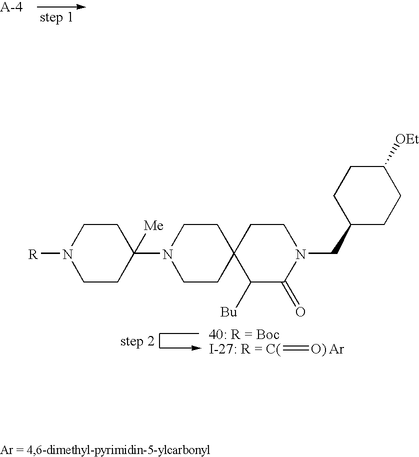

4-Ethoxy-cyclohexylmethyl p-toluenesulfonate can be prepared from 4-ethoxy-cyclohexane carboxylic by reduction with a hydride reducing agent and treating the resulting alcohol with toluenesulfonyl chloride and pyridine.

Step 1—To a mixture of A-4 (0.2 g, 0.474 mmol) and NaH (19 mg, 0.474 mmol) in a microwave vial was added NMP (1 mL) and the mixture was stirred for 10 min. To the mixture was added 4-ethoxy-cyclohexylmethyl p-toluenesulfonate. The reaction mixture was irradiated in a microwave and heated to 150° C. for 1 h. The reaction was not complete and the following data heating was continued at 150° C. for an additional 3 h. The resulting solution was diluted with saturated NH₄Cl and the solution extracted with ether (6×20 mL). The combined extracts were was with water and brine. The solvent was removed and the crude product purified by SiO2 chromatography eluting with a gradient of DCM and a 60:40:1 solution of DCM:MeOH:NH₄OH (100 to 80% DCM) which afforded 10 mg (ca. 3%) of 40 which was further purified on a preparative TLC developed with 5% MeOH/DCM. Additional material could be recovered form the aqueous phase.

Step 2—Removal of the Boc protecting group was carried out with TFA/DCM as described in step 7 of example 1. To a mixture of the crude amine (0.1 g, 0.216 mmol), EDCI (0.078 g, 3.68 mmol), HOBt (0.05 g, 0.368 mmol) and 4,6-dimethyl-pyrimidine-5-carboxylic acid was added DCM (3 mL) and DIPEA (0.19 mL, 1.08 mmol)). The solution was stirred at RT for 18 h. Saturated NaHCO₃ was added and the resulting mixture extracted with DCM (5×30 mL). The combined extracts were dried (MgSO₄), filtered and concentrated. The crude product was purified by SiO₂ chromatography eluting with a gradient of DCM and a 60:40:1 solution of DCM:MeOH:NH₄OH (100 to 70% DCM) which afforded 0.060 g of 1-27. An additional 0.069 g was obtained from purification of impure fraction by preparative TLC. The combined product was dissolved in 10% HCl/MeOH (6 mL) and the solution evaporated to afford the tris-hydrochloride salt: calcd. for C₃₅H₅₇N₅O₃.3HCl.1.2H₂O: C, 57.8; H, 8.65; N, 9.64. Found: C, 57.79, H, 8.34, N, 9.70.

The following compounds are prepared by analogous procedures except in step 2,4,6-dimethyl-pyrimidine-5-carboxylic acid is replaced with the carboxylic acid is parentheses: I-28 (2,4-dimethyl-6-oxo-6H-pyran-3-carboxylic acid, CASRN 480-65-9), I-29 (2,4-dimethyl-6-oxo-1,6-dihydro-pyridine-3-carboxylic acid, CASRN 5262105), I-30 (3,5-dimethyl-isoxazole-4-carboxylic acid, CASRN 2510-36-3) and I-31 (6-cyano-2,4-dimethyl-nicotinic acid, CASRN 871492-97-6).

### EXAMPLE 9

### (S)-1-Butyl-3-(4-hydroxy-cyclohexylmethyl)-9-[4-methyl-1-(3-methyl-5-trifluoromethyl-isoxazole-4-carbonyl)-piperidin-4-yl]-3,9-diaza-spiro[5.5]undecan-2-one (I-33)

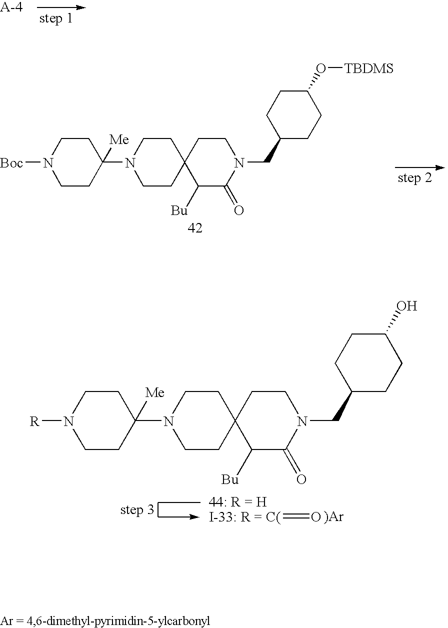

4-(tert-butyl-dimethyl-silanyloxy)-cyclohexylmethyl toluene-4-sulfonate (41) can be prepared by treating [4-(tert-butyl-dimethyl-silanyloxy)-cyclohexyl]-methanol with p-toluenesulfonyl chloride in pyridine under standard conditions.

Step 1—A mixture of A-4 (0.40 g, 0.95 mmol), 41 (0.850 g), K₂CO₃ (0.132 g, 0.95 mmol), tetra-butylammonium bromide (0.308 g), NaOH (0.040 g, 4.0 mmol) and toluene (1.6 mL) were mixed in a 5 mL flas which was then filled to the neck with toluene. The mixture was stirred vigorously (800 revolutions/min) for 40 h, then the reaction mixture was partitioned between water and EtOAc. The organic layer was twice washed with brine, dried (MgSO₄), filtered and evaporated. The crude product was purified by SiO₂ chromatography eluting with a MeOH/DCM gradient (1.5 to 4.5% MeOH containing about 0.25% NH₄OH) to afford 200 mg of 42.

Step 2—The Boc protecting group was removed with 4M HCl/dioxane as in step 2 of example 4 which resulted in concomitant removal of the silyl group.

Step 3—The resulting amine (97 mg), EDCI (40 mg), HOBt (28 mg), DIPEA (0.1 mL) and DCM (3 mL). An 1.5 mL aliquot of the resulting solution was added to 3-methyl-5-trifluoromethyl-isoxazole-4-carboxylic acid (13 mg) and the resulting solution was stirred overnight at RT. The reaction was partitioned between EtOAc and water and the EtOAC extract washed with brine, dried (MgSO₄), filtered and evaporated. The crude product was purified on a SiO₂ preparative TLC plate developed with 5% MeOH/DCM/0.25% NH₄OH to afford 21 mg of I-33.

I-32 and I-34 can be prepared analogously except in step 3,3-methyl-5-trifluoromethyl-isoxazole-4-carboxylic acid is replaced with 4,6-dimethyl-pyrimidine-5-carboxylic acid and 6-cyano-2,4-dimethyl-nicotinic acid respectively.

### EXAMPLE 10 (REFERENTIAL EXAMPLE)

### Chiral Synthesis

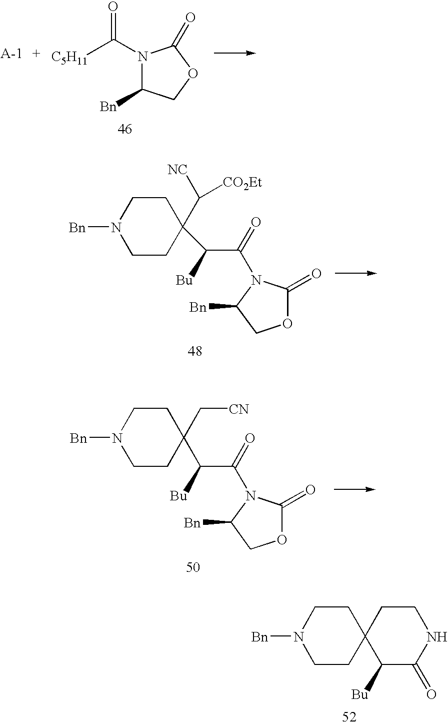

Step 1—To a solution of diisopropyl amine (6.2 mL) and THF (100 mL) maintained under an Ar atmosphere and cooled to −78° C. was added n-BuLi. The solution was stirred for 20 min, warmed to RT for 20 min than recooled to −78° C. A solution of 46 (11.493 g) and THF (25 mL) was added dropwise over a 10 min period and the resulting reaction stirred at −78° C. for 50 min. A solution of A-1 (11.87 g) and THF (25 mL) was added over 10 min then stirred at −78° C. for 1 h. The reaction mixture was partitioned between EtOAc (400 mL) and NaHCO₃ (400 mL) then stirred overnight. The layers were separated and the aqueous phase was washed with EtOAc and the combined extracts were dried (MgSO₄), filtered and evaporated. The crude product was purified by SiO2 chromatography eluting with a EtOAc/hexane gradient (20 to 30% EtOAc) to afford 19.612 g (84%) of 1-benzyl-4-[(S)-1-((R)-4-benzyl-2-oxo-oxazolidine-3-carbonyl)-pentyl]-piperidin-4-yl}-cyano-acetic acid ethyl ester (48) as a white foam.

Step 2—A solution of 48 (19.60 g, 35.01 mmol) in DMSO (120 mL) and water (3 mL) was heated in an oil bath to 135° C. and LiCl (42.39 g) was added. The internal temperature was warmed to 134° C. and stirred for 30 min (43 min total after addition of LiCl). The reaction was cooled to RT and partitioned between EtOAc and water. The aqueous layer was twice extracted with EtOAc and the combined extracts were dried (MgSO₄), filtered and evaporated. The crude product was purified by SiO₂ chromatography eluting with EtOAc/hexane (20 to 30% EtOAC) to afford 12.19 g (71%) of 50.

Step 3—To a solution of 50 (12.18 g) in MeOH (135 mL) at RT was added CoCl₂.6H₂O (11.5 g) followed by portionwise addition of NaBH₄ (9.26 g). The mixture was stirred at RT for 1.5 h. The reaction mixture was concentrated in vacuo and the resulting black mass was dissolved in 1N HCl. EtOAc was added followed by 28% NH₄OH to adjust the pH to 14. The resulting solution was stirred overnight then filtered through CELITE and the pad of CELITE washed with EtOAc. The aqueous layer was washed with EtOAc and the combined organic extracts were dried (MgSO₄), filtered and evaporated. The residue was purified on a SiO₂ column eluting with a stepwise gradient of DCM and a solution containing 10% MeOH/DCM/0.5% NH₄OH (100, 85, 75 to 55% DCM) to afford 5.89 g of (S)-9-benzyl-1-butyl-3,9-diaza-spiro[5.5]undecan-2-one (52).

(R)-9-Benzyl-1-butyl-3,9-diaza-spiro[5.5]undecan-2-one was prepared in identical fashion utilizing (S)-4-benzyl-3-hexanoyl-oxazolidin-2-one in place of 46. One skilled in the art will appreciate that these chiral intermediates can be substituted for the racemic compound A-3a.

### EXAMPLE 11

### CCR5-Mediated CCF Assay

CCF assay was performed as described before (C. Ji, J. Zhang, N. Cammack and S. Sankuratri, J. Biomol. Screen. 2006 11(6):652-663). Hela-R5 cells (express gp160 from R5-tropic virus and HIV-1 Tat) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/mL G418, 100 μg/mL hygromycin, and 1 μg/mL Doxycycline (Dox) (BD Bioscience, Palo Alto, Calif.), using Multimek (Beckman, Fullerton, Calif.) and incubated at 37° C. overnight to induce the expression of gp160. Ten μL diluted compounds in medium containing 5% DMSO were added to the cells, followed by the addition of CEM-NKr-CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μL/well and incubated for 24 hrs. At the end of co-culture, 15 μL of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% DMSO (CalBiochem, La Jolla, Calif.) and 1× Pen-Strep5 μL each of the two diluted compound or mAb to be tested for drug-drug interactions were added to the Hela-R5 cells right before the addition of target cells. Representative results are compiled in TABLE II

TABLE II

| | Cell-Cell Fusion (CCF) Assay |
|---|---|
| Cpd. No. | IC₅₀ (μM) |
| I-3 | 0.0095 |
| I-19 | 0.0078 |
| I-25 | 0.0098 |

### EXAMPLE 12

### Antiviral Assay

The sensitivity of a recombinant HIV-1 virus pseudotyped with the envelope proteins of the CCR5-tropic virus NLBal to test compounds was determined in a Luciferase reporter assay using JC53BL cells. NLBal pseudotyped HIV-1 was generated by calcium phosphate transfection of 293T cells (ATCC) with equal amounts of DNA of an envelope-deleted HIV-1 plasmid and of a NLBal envelope expression plasmid. The media (DMEM, 10% fetal bovine serum, 1% Penicillin/streptomycin, 1% Glutamine, all Gibco) was changed 16 h post-transfection and virus containing supernatant was harvested 48 h post-transfection. To determine the sensitivity of NLBal pseudotyped HIV-1, 25.000 JC53BL cells (NIH AIDS Reagent Program) were infected with NLBal pseudotyped HIV-1 in presence of a drug gradient in white 96 well plates (Greiner Bio-one). The volume was adjusted to 200 μL using assay media (DMEM, 10% fetal bovine serum, 1% Penicillin/streptomycin, 1% Glutamine). After incubation at 37° C., 90% relative humidity, 5% CO₂ for 3 days, 50 μL of Steady-Glo® Luciferase reagent (Promega) was added, incubated for 5 min at RT and luminescence was measured using a luminometer (Luminoskan, Thermo). The 50% and 90% inhibitory concentrations were calculated using Microsoft XL Fit 4 software. Representative results are compiled in TABLE III.

TABLE III

| | Antiviral Assay |
|---|---|
| Cpd. No. | IC₅₀ (μM) |
| I-5 | 0.0042 |
| I-6 | 0.0045 |
| I-22 | 0.0068 |

### EXAMPLE 13

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I wherein: 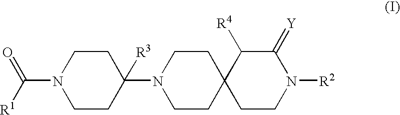
R¹ is selected from the group consisting of (i)-(v) and (vi): 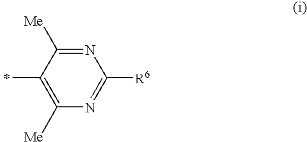
wherein:
R⁶ is hydrogen, C₁₋₃ haloalkyl, —N(R^{a})-A¹-C(O)R⁷ or —NR^{b}R^{c};
R^{a} is hydrogen or C₁₋₃ alkyl;
A¹ is C₁₋₆ straight or branched alkylene;
R^{b} and R^{c} are (A) together a group (CH₂)₂X¹(CH₂)₂, or, (B) independently R^{a} is hydrogen or C₁₋₃ alkyl and R^{b} is hydrogen, C₁₋₃ alkyl, C₁₋₃ acyl; —SO₂C₁₋₆ alkyl or C₁₋₆ hydroxyalkyl; 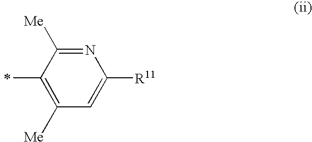
wherein:
R¹¹ is hydrogen, C₃₋₆ cycloalkyl, cyano, OR^{6a} or —O-A¹-C(O)R⁷;
A¹ is C₁₋₆ straight or branched alkylene;
R^{6a} is hydrogen, C₁₋₃ alkyl or C₁₋₆ hydroxyalkyl; 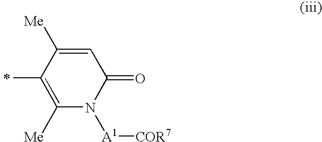
wherein:
A¹ is C₁₋₆ straight or branched alkylene; 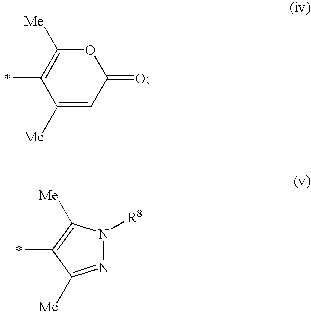
wherein:
R⁸ is C₃₋₇ cycloalkyl, (CH₂)ₙCOR⁷, —OR¹⁵, heteroaryl selected from the group consisting of pyridine, pyrimidine, pyrazine and pyridazine said heteroaryl optionally substituted with C₁₋₃ alkyl or C₁₋₃ haloalkyl;
n is 1 to 3;
R¹⁵ is hydrogen or C₁₋₆ alkoxy; 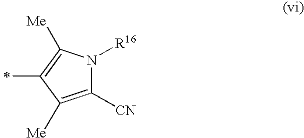
wherein:
R¹⁶ is hydrogen, hydroxyl or C₁₋₆ alkoxy; 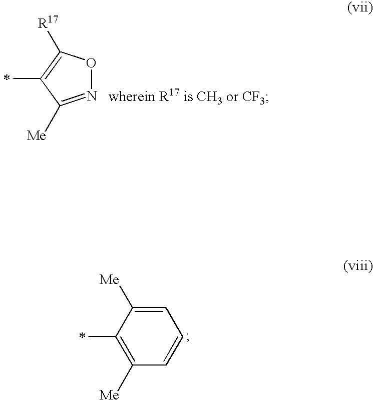
R² is A²-R⁹;
R³ is hydrogen or C₁₋₆ alkyl;
R⁴ is C₁₋₆ alkyl, C₁₋₆ alkoxy or phenyl;
R⁷ is hydroxy, C₁₋₆ alkoxy or NR^{e}R^{f};
A² is (CH₂)ₙ, C(O) or S(O)₂ wherein n is an integer from zero to three;
Y is O or H,H;
R⁷ is hydroxyl, NR^{e}R^{f}, or C₁₋₆ alkoxy;
R^{e} and R^{f} are (A) together a group (CH₂)₂X¹(CH₂)₂, or, (B) R^{e} and R^{f} are independently hydrogen or C₁₋₃ alkyl;
R⁹ is:
(a) C₃₋₆ cycloalkyl wherein said cycloalkyl is optionally substituted with one to three groups independently selected from the group consisting of OR¹⁴, C₁₋₃ alkyl, oxo, halogen and NR¹²R¹³ wherein R¹⁴ is hydrogen, C₁₋₆ alkyl, C₁₋₃ alkoxy-C₁₋₆ alkyl, carbamoyl, C₁₋₃ alkylcarbamoyl or C₁₋₃ dialkylcarbamoyl, R¹² is C₁₋₆ alkylsulfonyl, C₁₋₆ alkoxycarbonyl or C₁₋₆ acyl and R¹³ is hydrogen or C₁₋₆ alkyl;
(b) heterocyclyl selected from the group consisting of tetrahydropyranyl, tetrahydrofuranyl, oxetanyl, [1,4]dioxanyl, 3-oxa-bicyclo[3.1.0]hex-6-yl or hexahydro-furo[2,3-b]furan-3-yl said heterocycle optionally substituted with one or two C₁₋₃ alkyl; 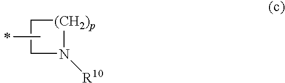
wherein;
p is an integer from one to three;
R¹⁰ is C₁₋₆ acyl, C₁₋₆ alkoxycarbonyl, C₁₋₆ alkyl-SO₂, C₁₋₆ haloalkyl, C₃₋₆ cycloalkyl;
(d) *—NR^{g}R^{h} wherein:
(A) R^{g} and R^{h} together are (CH₂)₂X¹(CH₂)₂, (B) together with the nitrogen to which they are attached are C₃₋₅ alkylene optionally substituted by C₁₋₃ alkyl, C₁₋₃ alkoxy, C₁₋₃ hydroxyalkyl or hydroxy, or, (C) taken independently R^{g} is hydrogen or C₁₋₃ alkyl and R^{h} is hydrogen, C₁₋₃ alkyl, C₁₋₃ acyl; —SO₂C₁₋₆ alkyl or C₁₋₆ hydroxyalkyl;
(e) *—OR^{j} wherein R^{j} is C₁₋₆ alkyl or tetrahydropyran-4-yl;
(f) C₁₋₁₀ alkyl;
(g) C₁₋₁₀ heteroalkyl;
(h) phenyl;
(i) pyridinyl;
(j) pyrazol-4-yl;
(k) imidazolyl;
wherein said phenyl, pyridinyl, pyrazol-4-yl or imidazolyl are optionally independently substituted with one to three groups independently selected from C₁₋₃ alkyl, C₁₋₃ alkoxy, C₃₋₆ cycloalkyl, halogen, C₁₋₆ alkoxycarbonyl, carbamoyl, —C₁₋₆ alkyl carbamoyl, di-C₁₋₆ alkyl carbamoyl, C₁₋₆ alkylsulfanyl, C₁₋₆ alkylsulfinyl or C₁₋₆ alkylsulfonyl, amino, C₁₋₃ alkylamino or C₁₋₃ dialkylamino;
X¹ is O, S(O)ₘ, NR^{d};
R^{d} is hydrogen, C₁₋₃ alkyl, C₁₋₃ acyl, C₁₋₆ alkylsulfonyl;
m is zero to two;
or, pharmaceutically acceptable salts thereof.

2. A compound according to claim 1 wherein:
R¹ is (i) wherein R⁶ is hydrogen or (ii) wherein R¹¹ is cyano;
R³ is methyl;
R⁴ is n-C₄H₉;
A² is SO₂; and,
R⁹ is C₁₋₆ alkyl, C₃₋₆ cycloalkyl, phenyl, pyridinyl, tetrahydropyranyl, tetrahydrofuranyl, or NR^{g}R^{h}.

3. A compound according to claim 2 wherein R⁹ is methyl, cyclopropyl, phenyl, 2-pyridinyl or NR^{g}R^{h}; and, R^{g} and R^{h} are independently are hydrogen or C₁₋₃ alkyl or together are (CH₂)₂X¹(CH₂)₂.

4. A compound according to claim 3 wherein R⁹ is NR^{g}R^{h} and X¹ is O.

5. A compound according to claim 1 wherein:
R¹ is (i) wherein R⁶ is hydrogen or (ii) wherein R¹¹ is cyano;
R³ is methyl;
R⁴ is n-C₄H₉;
A² is SO₂; and,
R⁹ is (c) 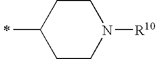
wherein R¹⁰ is C₁₋₆ acyl, C₁₋₆ alkoxycarbonyl, C₁₋₆ alkyl-SO₂, C₁₋₆ haloalkyl.

6. A compound according to claim 1 wherein:
R¹ is (i) wherein R⁶ is hydrogen or (ii) wherein R¹¹ is cyano;
R³ is methyl;
R⁴ is n-C₄H₉;
A² is CH₂; and,
R⁹ is tetrahydropyran-4-yl, 4-C₁₋₆ alkoxy-cyclohexyl, 4-fluorophenyl, or: 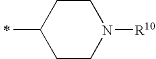
wherein R¹⁰ is C₁₋₆ acyl, C₁₋₆ alkoxycarbonyl, C₁₋₆ alkyl-SO₂, C₁₋₆ haloalkyl.

7. A compound according to claim 1 wherein:
R¹ is (i) wherein R⁶ is hydrogen or (ii) wherein R¹¹ is cyano;
R³ is methyl;
R⁴ is n-C₄H₉;
A² is C(O); and,
R⁹ is tetrahydropyran-4-yl or 4-oxa-tetrahydropyran.

8. A compound according to claim 1 selected from the group consisting of:
{1-Butyl-9-[1-(4,6-dimethyl-pyrimidine-5-carbonyl)-4-methyl-piperidin-4-yl]-3,9-diaza-spiro[5.5]undec-3-yl}-(tetrahydro-pyran-4-yl)-methanone;
5-{4-[7-Butyl-9-(tetrahydro-pyran-4-carbonyl)-3,9-diaza-spiro[5.5]undec-3-yl]-4-methyl-piperidine-1-carbonyl}-4,6-dimethyl-pyridine-2-carbonitrile;
[4-(9-Benzenesulfonyl-7-butyl-3,9-diaza-spiro[5.5]undec-3-yl)-4-methyl-piperidin-1-yl]-(4,6-dimethyl-pyrimidin-5-yl)-methanone;
1-Butyl-9-[1-(4,6-dimethyl-pyrimidine-5-carbonyl)-4-methyl-piperidin-4-yl]-3-(tetrahydro-pyran-4-ylmethyl)-3,9-diaza-spiro[5.5]undecan-2-one;
1-Butyl-9-[1-(4,6-dimethyl-pyrimidine-5-carbonyl)-4-methyl-piperidin-4-yl]-3-(4-fluoro-benzyl)-3,9-diaza-spiro[5.5]undecan-2-one;
5-{4-[7-Butyl-8-oxo-9-(tetrahydro-pyran-4-ylmethyl)-3,9-diaza-spiro[5.5]undec-3-yl]-4-methyl-piperidine-1-carbonyl}-4,6-dimethyl-pyridine-2-carbonitrile;
{4-[7-Butyl-9-(pyridine-2-sulfonyl)-3,9-diaza-spiro[5.5]undec-3-yl]-4-methyl-piperidin-1-yl}-(4,6-dimethyl-pyrimidin-5-yl)-methanone;
[4-(7-Butyl-9-cyclopropanesulfonyl-3,9-diaza-spiro[5.5]undec-3-yl)-4-methyl-piperidin-1-yl]-(4,6-dimethyl-pyrimidin-5-yl)-methanone;
1-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one;
[4-(7-Butyl-9-methanesulfonyl-3,9-diaza-spiro[5.5]undec-3-yl)-4-methyl-piperidin-1-yl]-(4,6-dimethyl-pyrimidin-5-yl)-methanone;
1-Butyl-9-[1-(4,6-dimethyl-pyrimidine-5-carbonyl)-4-methyl-piperidin-4-yl]-3,9-diaza-spiro[5.5]undecane-3-carboxylic acid tetrahydro-pyran-4-yl ester;
1-Butyl-9-[1-(4,6-dimethyl-pyrimidine-5-carbonyl)-4-methyl-piperidin-4-yl]-3,9-diaza-spiro[5.5]undecane-3-sulfonic acid dimethylamide;
{4-[7-Butyl-9-(tetrahydro-pyran-4-sulfonyl)-3,9-diaza-spiro[5.5]undec-3-yl]-4-methyl-piperidin-1-yl}-(4,6-dimethyl-pyrimidin-5-yl)-methanone;
{4-[7-Butyl-9-(1-methyl-1H-imidazole-4-sulfonyl)-3,9-diaza-spiro[5.5]undec-3-yl]-4-methyl-piperidin-1-yl}-(4,6-dimethyl-pyrimidin-5-yl)-methanone;
5-{4-[7-Butyl-9-(1-difluoromethyl-3,5-dimethyl-1H-pyrazole-4-sulfonyl)-3,9-diaza-spiro[5.5]undec-3-yl]-4-methyl-piperidine-1-carbonyl}-4,6-dimethyl-pyridine-2-carbonitrile;
{4-[7-Butyl-9-(1-difluoromethyl-3,5-dimethyl-1H-pyrazole-4-sulfonyl)-3,9-diaza-spiro[5.5]undec-3-yl]-4-methyl-piperidin-1-yl}-(4,6-dimethyl-pyrimidin-5-yl)-methanone;
{4-[7-Butyl-9-(tetrahydro-pyran-4-ylmethyl)-3,9-diaza-spiro[5.5]undec-3-yl]-4-methyl-piperidin-1-yl}-(4,6-dimethyl-pyrimidin-5-yl)-methanone;
{4-[7-Butyl-9-(morpholine-4-sulfonyl)-3,9-diaza-spiro[5.5]undec-3-yl]-4-methyl-piperidin-1-yl}-(4,6-dimethyl-pyrimidin-5-yl)-methanone;
[4-(9-Cyclopropanesulfonyl-7-propoxy-3,9-diaza-spiro[5.5]undec-3-yl)-4-methyl-piperidin-1-yl]-(4,6-dimethyl-pyrimidin-5-yl)-methanone;
4-{1-Butyl-9-[1-(4,6-dimethyl-pyrimidine-5-carbonyl)-4-methyl-piperidin-4-yl]-2-oxo-3,9-diaza-spiro[5.5]undec-3-ylmethyl}-piperidine-1-carboxylic acid methyl ester;
1-Butyl-3-[1-(2,2-difluoro-ethyl)-piperidin-4-ylmethyl]-9-[1-(4,6-dimethyl-pyrimidine-5-carbonyl)-4-methyl-piperidin-4-yl]-3,9-diaza-spiro[5.5]undecan-2-one;
1-Butyl-9-[1-(6-cyano-2,4-dimethyl-pyridine-3-carbonyl)-4-methyl-piperidin-4-yl]-3,9-diaza-spiro[5.5]undecane-3-carboxylic acid tetrahydro-pyran-4-yl ester;
1-Butyl-9-[1-(6-cyano-2,4-dimethyl-pyridine-3-carbonyl)-4-methyl-piperidin-4-yl]-3,9-diaza-spiro[5.5]undecane-3-sulfonic acid dimethylamide;
4-{1-Butyl-9-[1-(4,6-dimethyl-pyrimidine-5-carbonyl)-4-methyl-piperidin-4-yl]-3,9-diaza-spiro[5.5]undecane-3-sulfonyl}-piperidine-1-carbaldehyde;
1-(4-{1-Butyl-9-[1-(4,6-dimethyl-pyrimidine-5-carbonyl)-4-methyl-piperidin-4-yl]-3,9-diaza-spiro[5.5]undecane-3-sulfonyl}-piperidin-1-yl)-ethanone; and,
[4-((R)-7-Butyl-9-methanesulfonyl-3,9-diaza-spiro[5.5]undec-3-yl)-4-methyl-piperidin-1-yl]-(4,6-dimethyl-pyrimidin-5-yl)-methanone.

9. A method for treating or preventing an human immunodeficiency virus (HIV-1) infection, or treating AIDS or ARC, in a patient in need thereof which comprises administering to the patient in need thereof a therapeutically effective amount of a compound according to claim 1.

10. A method according to claim 9 further comprising co-administering a therapeutically effective amount of one or more compound(s) selected from the group consisting of HIV-1 nucleoside reverse transcriptase inhibitors, non-nucleoside reverse transcriptase inhibitors, HIV-1 protease inhibitors and HIV-1 viral fusion inhibitors and a compound of claim 1.

11. A method for treating an inflammatory disorder comprising administering a therapeutically effective amount of a compound of according to claim 1 to a patient in need thereof.

12. A method for according to claim 11 wherein the inflammatory disorder is rheumatoid arthritis.

13. A method according to claim 12 further comprising co-administering a therapeutically effective amount of one or more anti-inflammatory or analgesic compounds and a compound of claim 1.

14. A method for treating asthma or congestive obstructive pulmonary disease (COPD) comprising administering a therapeutically effective amount of a compound according to claim 1 to a patient in need thereof.

15. A method for treating solid organ transplant rejection comprising administering a therapeutically effective amount of a compound according to claim 1 to a patient in need.

16. A method according to claim 14 further comprising co-administering a therapeutically effective amount of one or more anti-rejection drugs or immunomodulators and a compound of claim 1.

17. A pharmaceutical composition comprising a compound according to claim 1 and at least one pharmaceutically acceptable carrier, diluent or excipient.
